(19) United States
(12) Patent Application Publication
(10) Pub. No.: US 2015/0147273 A1
(43) Pub. Date: May 28, 2015

(54) RADIOLABELED ANALOG(S) OF COMPOUND 0118 AND USE THEREOF IN CONNECTION WITH PET AND/OR SPECT IMAGING TO DETERMINE WHETHER A PHARMACEUTICAL CONTAINING COMPOUND 0118 IS A CANDIDATE CANCER TREATMENT FOR A PATIENT

(71) Applicant: KONINKLIJKE PHILIPS N.V., EINDHOVEN (NL)

(72) Inventors: Tilman Lappchen, Eindhoven (NL); Holger Gruell, Eindhoven (NL); Marc Stefan Robillard, Eindhoven (NL); Johan Lub, Valkenswaard (NL)

(73) Assignee: Koninklijke Philips N.V., Eindhoven (NL)

(21) Appl. No.: 14/404,657

(22) PCT Filed: Jun. 10, 2013
(86) PCT No.: PCT/IB2013/054744

§ 371 Date: Dec. 1, 2014

Related U.S. Application Data
(60) Provisional application No. 61/658,111, filed on Jun. 11, 2012.

(51) Int. Cl. A61K 51/04; A61B 6/03; C07D 249/04; A61K 31/4192; C07B 59/00; C07C 235/20

(52) U.S. Cl. CPC A61K 51/0453; C07B 59/002; C07C 235/20; C07D 249/04; A61K 31/4192; A61B 6/037
USPC 424/1.89; 548/255; 564/153; 600/425

(57) ABSTRACT

A method for determining whether compound 0118 is a candidate treatment for a patient includes processing, via a processor, image data of tissue of interest of a patient including a cancer to determine whether a radiolabeled analog of compound 0118 is present in the tissue of interest represented in the image data and generating a signal indicating that compound 0118 is a candidate treatment for the patient in response to the determining that the radiolabeled analog of compound 0118 is present in a predetermined amount in the tissue of interest represented in the image data, wherein the presence of the radiolabeled analog of compound 0118 in the tissue of interest indicates presence of a sub-type of cancer having a galectin-1 molecular target, which is a sub-type of treatable by compound 0118.

The following generally relates to radiotracers and is described in connection with Positron Emission Tomography (PET) and/or Single Photon Emission Computed Tomography (SPECT) imaging.

Molecular Imaging (MI) techniques have become an integral part of diagnostic procedures in the clinic, particularly in oncology. In addition to diagnostic applications, molecular imaging has also proven valuable for patient stratification, tumor staging, and therapy-monitoring, thereby becoming one of the hallmarks of personalized medicine.

While techniques such as Computer Tomography (CT) and Magnetic Resonance Imaging (MRI) provide high resolution anatomical images, nuclear imaging techniques such as SPECT and PET are highly sensitive techniques, which allow the detection of radioactively labeled molecules, the so-called ‘radiotracers’, in nano- to picomolar concentrations. To date, a variety of different radiotracers have been developed, which target disease-specific molecular markers and processes, e.g., spatial distribution, up- and/or down-regulation of certain receptors, and deviations from normal metabolic patterns. Apart from the growing significance for detection of neurodegenerative diseases, inflammation, and vascular diseases, oncology remains the key application area for nuclear imaging.

[¹⁸F]Fluorodeoxyglucose ([¹⁸F]FDG; glucose metabolism) is the most widely employed PET radiotracer worldwide, but the list of PET-radiotracers for oncology is steadily growing, alternative radiotracers being [¹⁸F]fluoride (bone scan), [¹⁸F]deoxyfluorothymidine ([¹⁸F]FLT; proliferation), [¹⁸F]fluoromisonidazole ([¹⁸F]FMISO; hypoxia), [¹¹C]choline (lipid metabolism), [¹¹C]methionine (amino acid metabolism), and others. Beside the traditional use for diagnostic procedures, nuclear imaging tracers increasingly gain importance for patient stratification and as companion diagnostics. For instance, radiotracers targeted towards specific molecular targets allow the selection of patients, which will benefit most from a certain type of therapy and are at lower risk for drug-specific toxicity, thereby contributing to the development of more effective and safer treatments.

Cancer management has not only progressed in the diagnostic area, but also in the therapeutic domain, where many new drugs were approved by the regulatory authorities and have become available for routine treatment in the clinic. Apart from the traditional cytostatics such as DNA-alkylating agents (cisplatin, chlorambucil, cyclophosphamide, etc.), antimetabolites (methotrexate, fluorouracil, floxuridine, etc.), DNA-cutters including topoisomerase poisons (bleomycine, daunorubicin, doxorubicin), DNA-binders (dactinomycin), and spindle poisons (vincristine, vinblastine, paclitaxel, docetaxel), the literature has indicate that nearly 900 new drugs and vaccines against cancer are currently in clinical trials. Many of these drugs are directed towards specific molecular targets, which are only present in certain (sub-)types of cancers, necessitating careful selection of the patient population potentially benefiting from a particular drug. This is particularly important for cancer therapies based on anti-hormones, such as anti-estrogens and anti-progestins, where expression of the corresponding receptor is a vital precondition for successful treatment. In addition, however, the same is true for most of the anti-angiogenic drugs currently on the market, in clinical trials and in preclinical development.

Anti-angiogenic treatment of cancer relies on depriving the fast-growing tumor cells from the required blood supply. Based on their mode of action, two main categories of anti-angiogenic drugs can be distinguished: directly acting angiostatic compounds, and angiogenesis inhibitors acting indirectly by blocking angiogenesis signalling. The latter may either occur by clearing pro-angiogenic growth factors from the circulation, by blocking their corresponding receptors, or by interfering with the downstream intracellular signaling pathways after receptor activation. The most prominent example of the first sub-class is Bevacizumab (Avastin®), the first FDA approved angiogenesis inhibitor, which is a humanized monoclonal antibody binding to vascular endothelial growth factor (VEGF), thereby preventing interaction with VEGF-receptors and suppressing endothelial cell proliferation and angiogenesis. Other FDA-approved monoclonal antibodies include trastuzumab (Herceptin®) targeting the HER2/neu receptor and cetuximab (Erbitux®), which binds to the extracellular domain of epidermal growth factor receptor (EGFR) and results in downregulation of VEGF expression. Finally, there is the growing number of small-molecule inhibitors of growth factor receptor tyrosine kinases, such as gefitinib (Iressa®), erlotinib (Tarceva®), vandetanib (Caprelsa®, ZD6474), and others.

In contrast to the indirectly acting anti-angiogenic drugs discussed above, directly acting angiostatic compounds have an effect on endothelial cells and regulatory pathways, which is independent of tumor cells. Among these compounds are, e.g., drugs inhibiting proliferation of endothelial cells (such as platelet factor-4 [PF4], endostatin) and drugs inhibiting extracellular matrix breakdown (such as inhibitors of matrix metalloproteinases, MMPs). Although clinical development of direct angiogenesis inhibitors is currently still lagging behind compared to indirect angiogenesis drugs, the latter class of compounds is expected to be less susceptible to drug-induced resistance. In the past decade, a library of β-sheet forming peptides has been designed based on the 3-dimensional structures of the bactericidal permeability-increasing protein (BPI) and α-chemokines interleukin-8 and PF4. One of these peptides, called anginex, showed particularly potent angio static activity.

Mechanistically, anginex was found to prevent attachment of activated endothelial cells to the extracellular matrix, ultimately leading to apoptosis. Notably, the cytotoxic effect of anginex proved to be specific for angiogenically activated endothelial cells (as those found in tumor vasculature), while resting endothelial cells (like those found in normal vasculature) were apparently not affected. Although the general mechanism of action was known shortly after discovery of anginex, the literature has indicated that it took about 5 years until galectin-1 was identified as its molecular target. Galectin-1 (gal-1) is overexpressed in endothelial cells of various tumors, and appears to be crucial for tumor angiogenesis. In addition, recent evidence suggests that angiogenic activation of endothelial cells may also occur via uptake of gal-1 secreted by tumor cells. Taken together, all these results underline the high potential of anginex and other gal-1-targeted angiostatic cancer therapies.

Although anginex and other anti-angiogenic peptides have shown promising anti-tumor effects in vivo, non-peptidic compounds are often superior drugs, mainly because they allow oral administration, generally lack an immune response, and display a better pharmacokinetic profile. Using the 3-dimensional molecular structure of anginex as a template, Dings, et al. “Design of nonpeptidic topomimetics of antiangiogenic proteins with antitumor activities,” J Natl Cancer Inst 98(13): 932-936, 2006, designed a small library of nonpeptidic, calix[4]arene based surface topomimetics, mimicking the spatial dimensions and the amphipathic nature of key amino acid side chains in anginex. One of these compounds, termed compound 0118, proved equipotent or even more potent than anginex both in in vitro assays of endothelial cell proliferation, endothelial cell migration, and angiogenesis, and in tumor growth models in vivo. In the meantime, compound 0118 has proven safe in toxicological studies and has already entered clinical studies.

Apart from the promising (pre)-clinical results obtained so far and the high expectations regarding development of compound 0118 into a pharmaceutical for anti-angiogenic cancer therapy, radio labelled derivatives of compound 0118 may prove highly valuable PET- and/or SPECT-imaging tracers for tumor diagnosis and/or for selection of patients amenable to treatment with compound 0118. However, such radiolabelled analogues of this compound are unknown. Unfortunately, design of radiolabelled analogues of compound 0118 is an intrinsically difficult task given the hitherto known structure-activity relationship (SAR) of a range of similar compounds, indicating that only minor modifications are tolerated without a significant loss of anti-angiogenic activity. Moreover, the high molecular weight of this drug molecule compared to other small molecule drugs imposes additional constraints on the design of a related radiotracer, as it is necessary to separate and remove unreacted precursor after radiolabeling in order to obtain a final radiotracer solution of high purity and high specific activity.

Aspects described herein address the above-referenced problems and/or others.

In one aspect, a method for determining whether compound 0118 is a candidate treatment for a patient includes processing, via a processor, image data of tissue of interest of a patient including a cancer to determine whether a radiolabeled analog of compound 0118 is present in the tissue of interest represented in the image data and generating a signal indicating that compound 0118 is a candidate treatment for the patient in response to the determining that the radiolabeled analog of compound 0118 is present in a predetermined amount in the tissue of interest represented in the image data, wherein the presence of the radiolabeled analog of compound 0118 in the tissue of interest indicates presence of a sub-type of cancer having a galectin-1 molecular target, which is a sub-type of treatable by compound 0118.

In another aspect, a method for monitoring treatment of cancer with compound 0118 includes processing image data of a treatment scan performed after at least one treatment with compound 0118 to determine whether a radio labeled analog of compound 0118 is present in tissue of interest represented in the image data, wherein the presence of the radio labeled analog of compound 0118 in the tissue of interest indicates presence of a sub-type of cancer having a galectin-1 molecular target, which is a sub-type of treatable by compound 0118, and generating and presenting a first recommendation signal recommending continuing treatment with compound 0118 in response to the image data of the treatment scan indicating the radio labeled analog of compound 0118 is present in a predetermined amount in the tissue of interest represented in the image data.

In another aspect, a computing system includes a radiotracer identifier that processes at least one of PET or SPECT image data and identifies a presence or absence of a predetermined amount of radiolabeled analog of compound 0118 in tissue of interest of a patient represented in the image data. The computing system further includes a recommender that generates and visually presents a first recommendation indicating that compound 0118 is a candidate treatment for the patient in response to the radiotracer identifier identifying the presence of the predetermined amount of radiolabeled analog of compound 0118 in tissue of interest of a patient represented in the image data.

In another aspect, a radiotracer includes an analog of compound 0118 and a radiolabel. In another aspect, a radiolabeled analog of compound 0118 includes a central calix[4]arene core and a hydrophobic substituent at an upper rim wherein the substituent is one of a radioiodinated or a radiobrominated derivative accessible via radiohalodestannylation from a corresponding tributylstannyl precursor. In another aspect, a radio labeled analog of compound 0118 includes a central calix[4]arene core and a hydrophilic substituent at a lower rim, wherein the substituent is an [¹⁸F]fluoroalkyltriazole moiety. In another aspect, a radio labeled analog of compound 0118 includes a central calix[4]arene core and a substituent at an equatorial position on a methylene bridge of the calix[4]arene core, wherein the substituent is from a group consisting of an [¹⁸F]fluoroalkyl chain or an [¹⁸F]fluoroalkyltriazole moiety.

In yet another aspect, non-radioactive analogues of compound 0118 containing a single substituent at an equatorial position of a methylene bridge within the calix[4]arene core can have therapeutic applications in antiangiogenic therapy. Specific applications include, but are not limited to, administration of therapeutically effective doses of the compounds to patients to achieve inhibition of progression or regression of various pathological conditions such as tumorigenesis, diabetic retinopathy, neovascular glaucoma, rheumatoid arthritis, restenosis, and diabetic retinopathy.

The invention may take form in various components and arrangements of components, and in various steps and arrangements of steps. The drawings are only for purposes of illustrating the preferred embodiments and are not to be construed as limiting the invention.

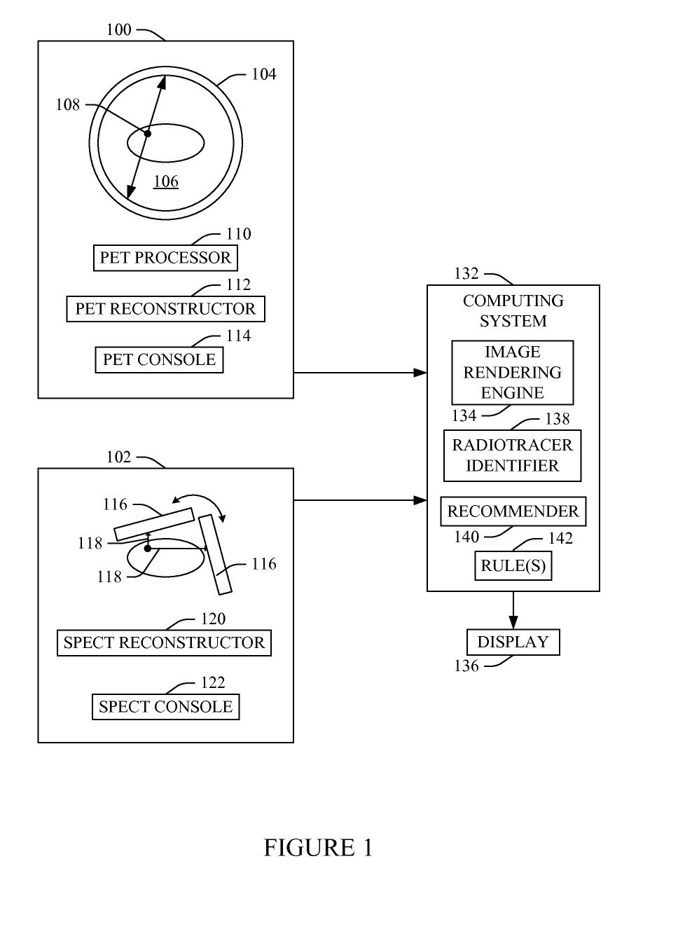

FIG. 1 schematically illustrates a computing system that processes image data from a PET and/or SPECT imaging system and identifies whether compound 0118 is a candidate treatment or still a candidate treatment for a patient.

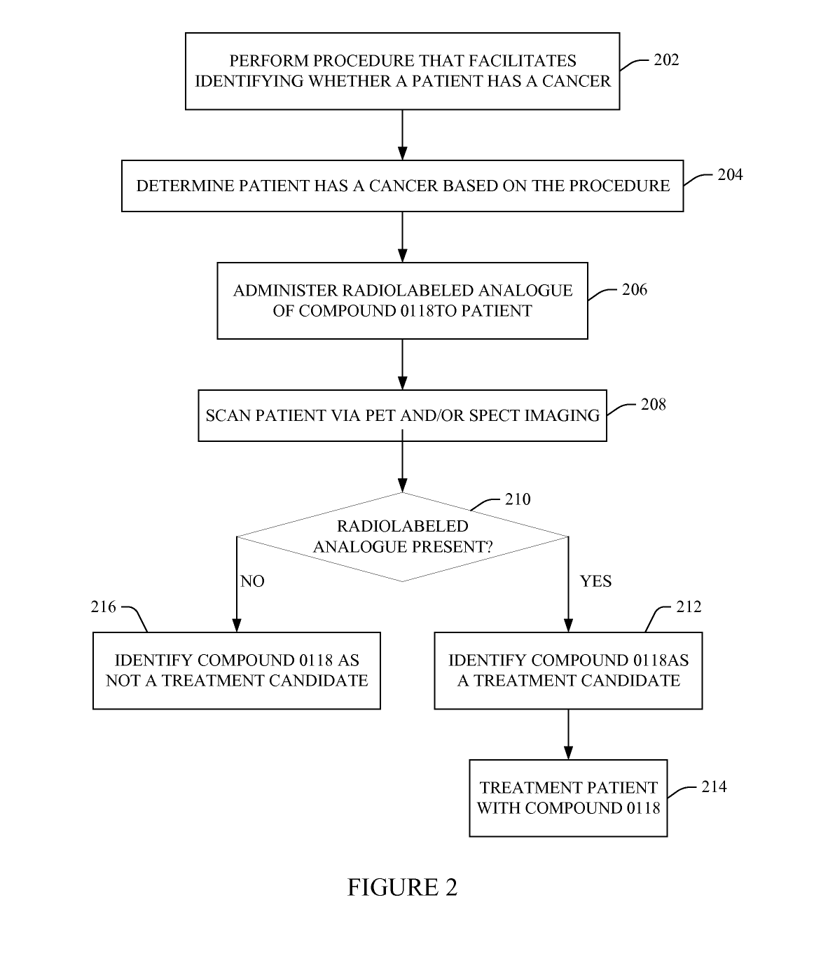

FIG. 2 illustrates an example method for identifying whether compound 0118 is a candidate as a treatment for a cancer for a patient.

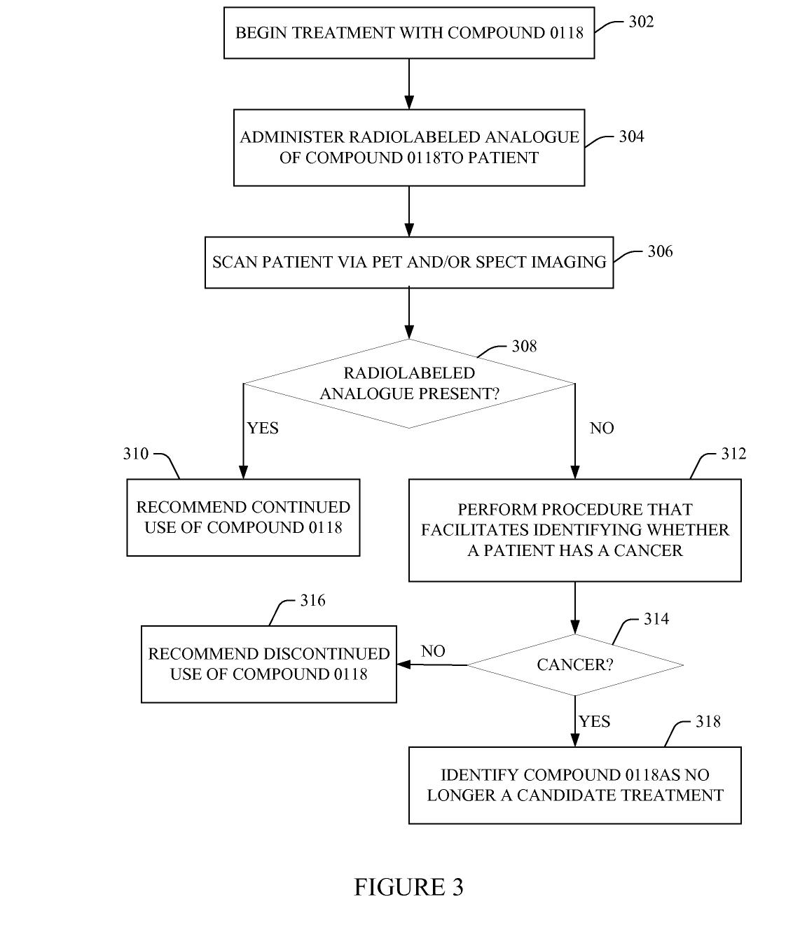

FIG. 3 illustrates an example method for monitoring treatment with compound 0118.

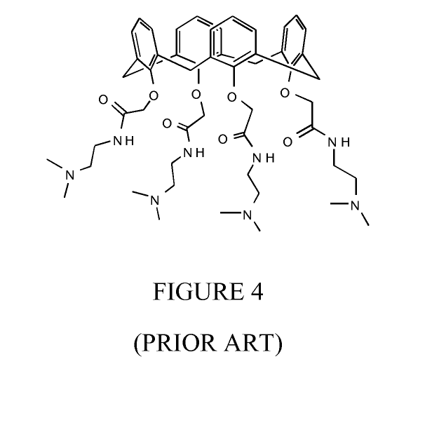

FIG. 4 illustrates a general structure of compound 0118.

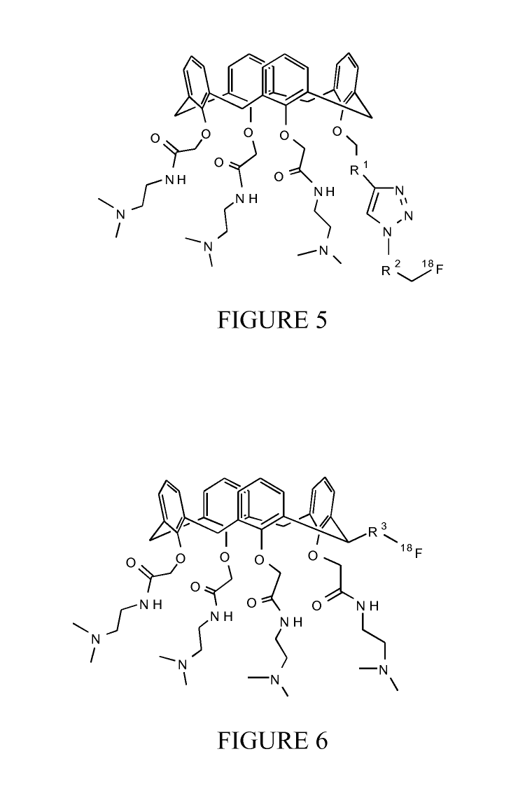

FIG. 5 illustrates a Class 1 radiolabeled analog of compound 0118. For example, R¹ and R² could be independently alkyl, cycloalkyl, heterocycloalkyl, aralkyl, heteroaralkyl, alkoxy, thioalkoxy, cycloalkylalkoxy, or heterocycloalkylalkoxy, and each of these groups could include halogen, hydroxyl, sulfhydryl, amide, ester, (poly)ether, phosphonate, sulfonate, and/or keto functionalities. Preferably, however, R¹ and R² are chosen from branched or linear alkyl- and/or (poly)-ether chains, such as polyethyleneglycol (PEG).

FIG. 6 illustrates a Class 2a radiolabeled analog of compound 0118. Among others, R³ may be chosen from alkyl, cycloalkyl, heterocycloalkyl, aralkyl, heteroaralkyl, alkoxy, thioalkoxy, cycloalkylalkoxy, or heterocycloalkylalkoxy, and each of these groups could include halogen, nitro, nitroso, keto, hydroxyl, sulfhydryl, amide, (poly)ether.

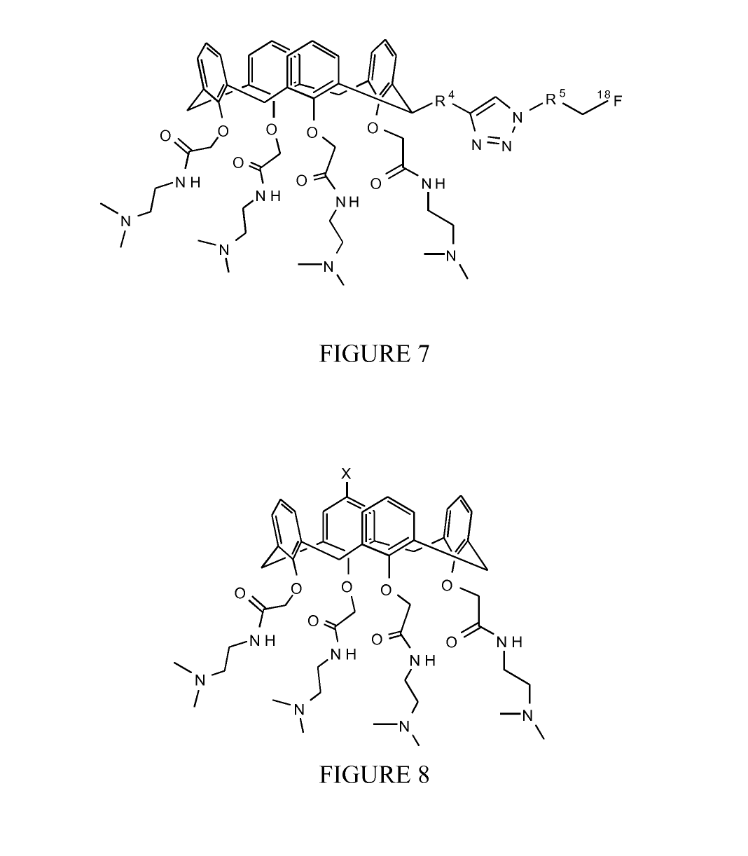

FIG. 7 illustrates a Class 2b radiolabeled analog of compound 0118. For example, R⁴ and R⁵ could be independently alkyl, cycloalkyl, heterocycloalkyl, aralkyl, heteroaralkyl, alkoxy, thioalkoxy, cycloalkylalkoxy, or heterocycloalkylalkoxy, and each of these groups could include halogen, hydroxyl, sulfhydryl, amide, ester, (poly)ether, phosphonate, sulfonate and/or keto functionalities. Preferably, however, R⁴ and R⁵ are chosen from branched or linear alkyl- and/or (poly)-ether chains, such as polyethyleneglycol (PEG).

FIG. 8 illustrates Class 3 radiolabeled analog of compound 0118. X denotes all radioactive isotopes of the halogens F, Cl, Br, I, At.

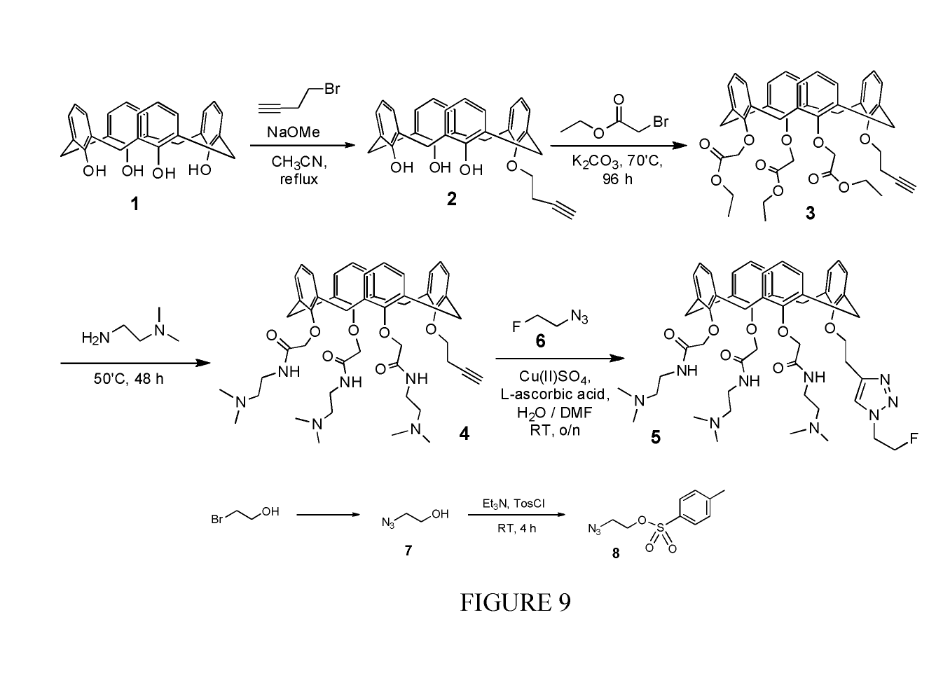

FIG. 9 illustrates a synthetic strategy for preparation of alkyne precursor 4 and non-radioactive reference compound 5 of a Class 1 radiotracer [¹⁸F]5.

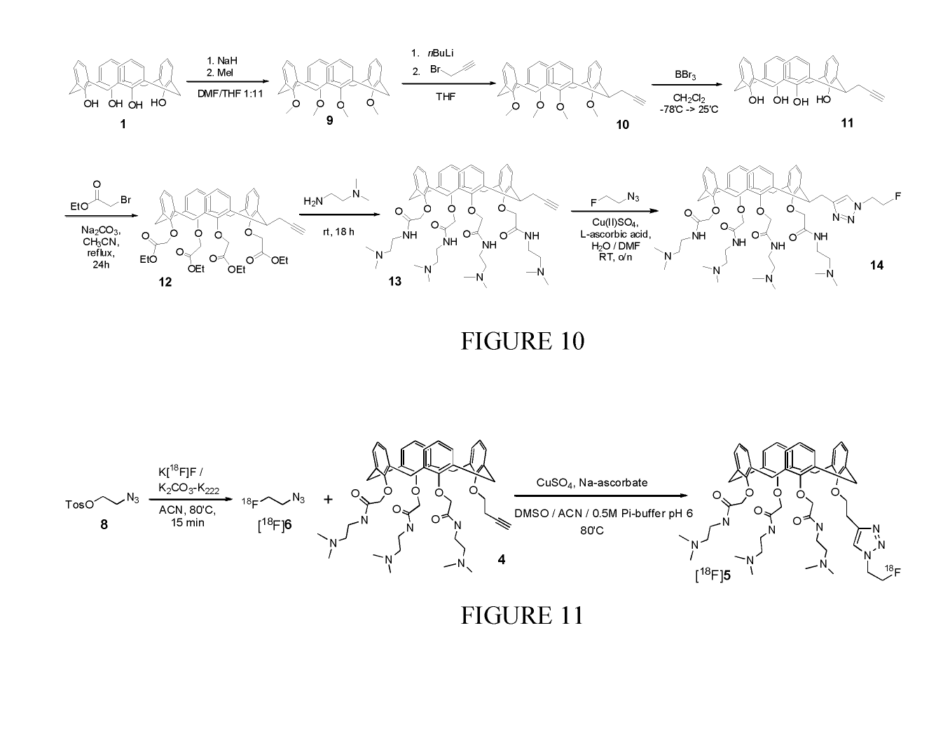

FIG. 10 shows a synthetic strategy towards alkyne precursor 13 and non-radioactive reference compound 14 of a Class 2b radiotracer [¹⁸F]14.

FIG. 11 shows Radiosynthesis of the [¹⁸F]-labelled compound 0118 analogue [¹⁸F]5

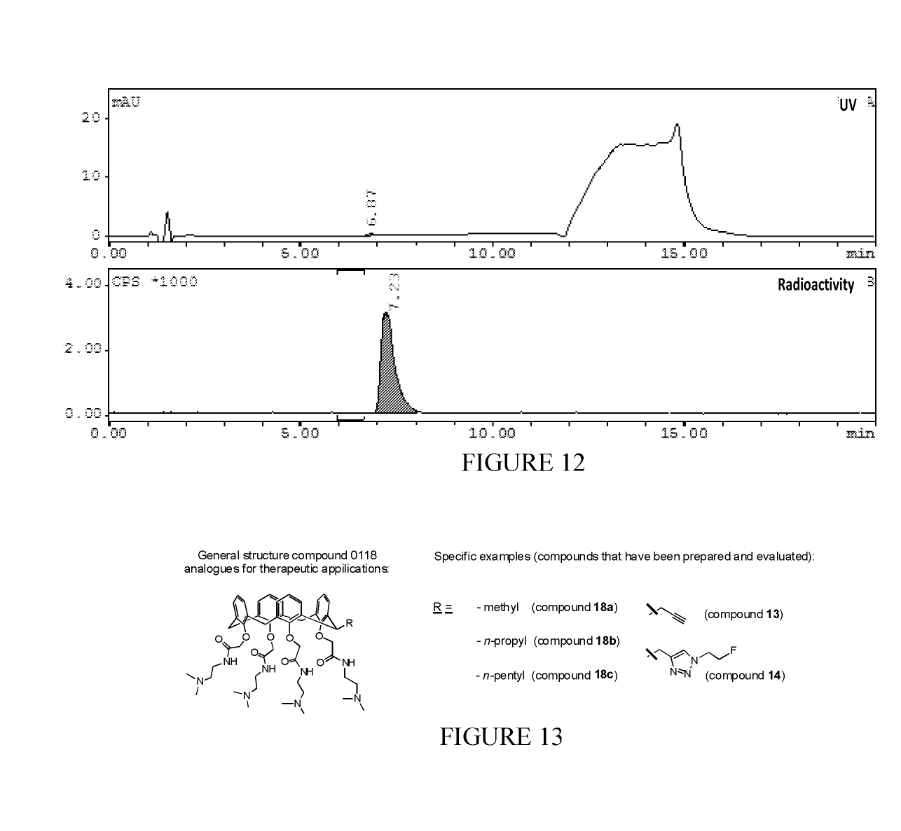

FIG. 12 illustrates analytical HPLC chromatogram of [¹⁸F]5 after purification by preparative HPLC.

FIG. 13 shows the general structure of non-radioactive analogues of compound 0118, which may be employed for anti-angiogenic therapy, including specific examples of compounds that have been prepared and characterized in terms of their anti-proliferative activity.

FIG. 14 shows a synthetic route towards compound 0118 analogues bearing a single equatorial methyl-, n-propyl, or n-pentyl substituent (specific examples of a new class of equatorially substituted 0118 analogues with general structure depicted in FIG. 13.

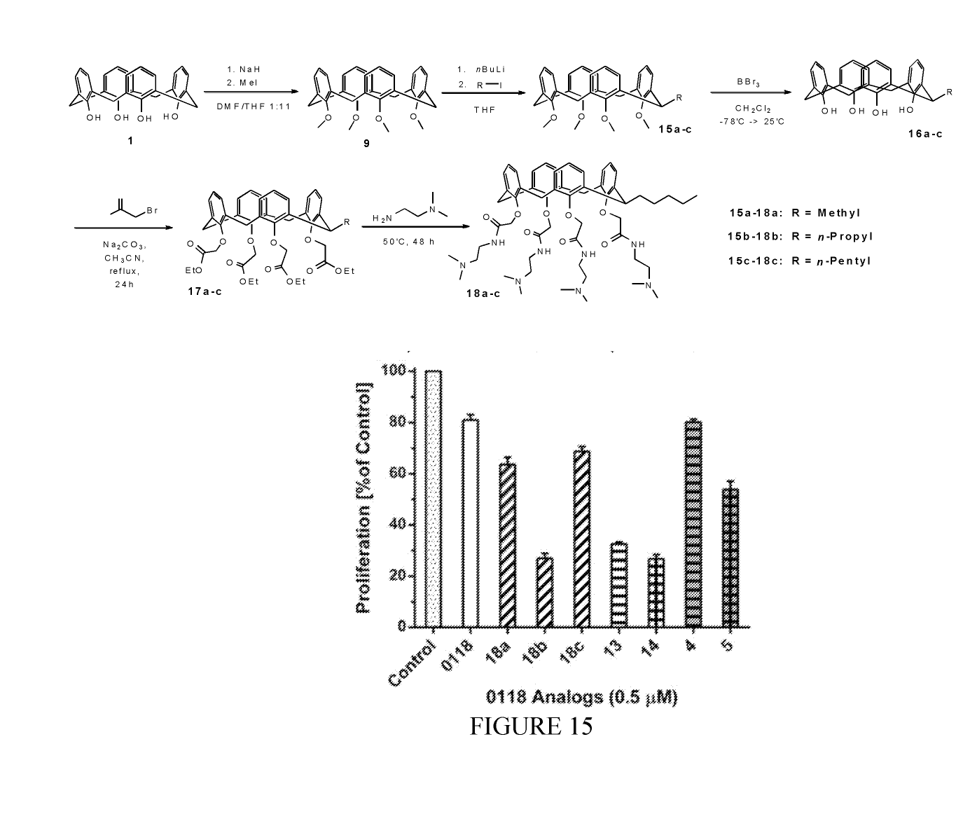

FIG. 15 illustrates the effect of lower-rim substituted 0118 analogues (compound 4, compound 5) and equatorially substituted 0118 analogues (compound 13, compound 14, compound 18a, compound 18b, and compound 18c) on the proliferation of mouse endothelial cells (2H11) with parent compound 0118 as reference.

Compound 0118 is a drug candidate for anti-angiogenic cancer therapy currently in clinical development for sub-types of cancer having a galectin-1 molecular target. Although highly desirable, up to now, radiolabelled analogues of this compound are unknown. Several such radiolabelled analogues are described in detail below. These radiotracers retain the anti-angiogenic activity of the parent compound and can be obtained in high radiochemical and chemical purity.

Image data generated by scanning a patient after administration of one of the radiolabelled analogues of compound 0118 via PET and/or SPECT imaging can be used to determine a presence, distribution, and/or location of the molecular target (galectin-1), which allows for identifying a sub-group of patients, from a population of patients, potentially benefiting from treatment of cancer with compound 0118, facilitate rendering a diagnosis of cancer with compound 0118 and/or monitoring cancer therapy with compound 0118.

FIG. 1 schematically illustrates a PET scanner 100 and a SPECT scanner 102.

The PET scanner 100 includes one or more rings of gamma radiation detectors 104 arranged around a PET examination region 106. The detector ring 104 detects 511 keV gamma rays produced in response to a positron annihilations event 108 occurring in the examination region 106. A PET processor 110 identifies coincident gamma pairs by identifying photons detected in temporal coincidence (or near simultaneously) along a line of response (LOR) and generates event by event or list mode data indicative thereof. The data may also include time-of-flight (TOF) information, which allows the location of an event along a LOR to be estimated. A PET reconstructor 112 reconstructs acquired PET data, generating a PET image. A PET console 114 allows a user to control the PET scanner 100.

The SPECT 102 imaging system includes one or more gamma radiation detectors 116 (two shown). The one or more gamma radiation detectors 116 detect gamma rays 118 emitted from a SPECT examination region and having energy in the diagnostic energy range (e.g., 40 to 140 keV). The gamma radiation detector 116 acquires projections from a number of angles with respect to the examination region by rotating the gamma radiation detector 116 around the examination region. A SPECT reconstructor 120 reconstructs the projections and produces volumetric data representative of the distribution of the radioisotope emitting the gamma rays in the object or subject. A SPECT console 122 allows a user to control the SPECT scanner 102.

A computing system 132 processes image data, including PET image data (e.g., from the PET scanner 100 and/or other PET scanner), SPECT image (e.g., from the SPECT scanner 102 and/or other SPECT scanner), and/or other image data. In one instance, this includes processing the image data to identify a presence (or absence) of a radiotracer taken up by cancer cells (e.g., FDG, which is taken up by high-glucose-using cells such as cancer cells or other tracer), presence (or absence) of one or more radiolabelled analogues of compound 0118, etc. For the later, this can be performed before and/or during cancer treatment with compound 0118.

The computing system 132 includes an image rendering engine 134, which renders PET, SPECT and/or other modality images via a display 136. Images from different modalities may be displayed concurrently and/or individually. In addition, the images may represent different points in time such as pre-treatment, treatment, and/or post-treatment images. Images acquired at different points in time can be to determine information (e.g., compute a value) that indicates whether a cancer has shrunk, grown or remained the same. In one instance, the image rendering engine 134 renders the image in an interactive graphical user interface (GUI), which includes tools for manipulating the image such as zoom, pan, rotate, segment, etc.

The computing system 132 further includes a radiotracer identifier 138 that identifies a presence (or absence) of one or more radiotracers from the image data. This may include determining a concentration, location, and/or distribution of an identified radiotracer. An example of such a radiotracer is a radiolabelled analog of compound 0118. The radiotracer identifier 138 generates a signal indicative of whether the radiotracer is present and/or the concentration, location, and/or distribution of an identified radiotracer. The signal can be presented as a notification via the display 136 in human readable form (e.g., text) with or without display of other information (e.g., an image) and/or conveyed to another device

The computing system 132 further includes recommender 140 that generates a recommendation signal based on the output of the radiotracer identifier 140 and a set of one or more predetermined rules 142. The rules 142 can be determined by clinicians, based on previous studies, and/or otherwise. As an example, a rule may indicate that a certain concentration, distribution, location, etc. is indicative of a presence or an absence of a radiotracer. The recommendation can be in the form of a signal that can be presented as a notification via the display 136 in human readable form (e.g., text) via the display 138 with or without display of other information (e.g., an image, radiotracer concentration, location, and/or distribution, etc.) and/or conveyed to another device.

By way of further non-limiting example, where the radiotracer identifier 140 identifies particular uptake of a radio labelled analog of compound 0118, after administration of the of radiolabelled analog of compound 0118, the recommender 142 may display a recommendation recommending compound 0118 as a candidate for cancer treatment or continued use of compound 0118 for cancer treatment. Where the radiotracer identifier 140 identifies particular absence of the radio labelled analog of compound 0118, after administration of the of radio labelled analog of compound 0118, the recommender 142 does not recommend compound 0118 as a candidate for cancer treatment (or recommends not using compound 0118 for cancer treatment) or recommends discontinuing use of compound 0118 as treatment for cancer.

The computing system 132 can be implemented via one or more processors executing computer readable instructions encoded, embedded, stored, etc. on computer readable storage medium such as physical memory. Additionally or alternatively, the computing system 132 can be implemented the one or more processors executing computer readable instructions in connection with other medium such as computer readable instructions carried by a signal, carrier wave and/or other transitory medium. In another embodiment, one or more of the components of the computing system 132 may be implemented in one or more other computing systems.

FIG. 2 illustrates an example method for identifying whether compound 0118 is a candidate as a treatment for a cancer for a patient.

At 202, a procedure that facilitates identifying whether a patient has a cancer is performed. The procedure may be a FDG-PET imaging procedure and/or procedures.

At 204, based on a result of the procedure, it is determined that the patient likely has a cancer. For example, the computing system 132, after processing FDG-PET images, may identify a presence of a level of FDG that indicates a likelihood of a cancer.

At 206, a radiolabeled analogue of compound 0118 is administered to the patient.

At 208, after a predetermined period of time to allow sufficient uptake of the radiolabeled analogue, the patient is scanned via a PET and/or SPECT imaging procedure.

At 210, it is determined whether the radiolabeled analogue is present in the image data.

If the radiolabelled analogue is present at a predetermined level, then at 212 compound 0118 is identified as a treatment candidate for the cancer for the patient.

At 214, compound 0118 is administered to the patient to treat the cancer.

If the radiolabelled analogue is not present, then at 216 compound 0118 is identified as not a treatment candidate for the cancer for the patient.

FIG. 3 illustrates an example method for monitoring a treatment with compound 0118.

At 302, treatment with compound 0118 begins for a patient identified as a candidate for cancer treatment with compound 0118.

At 304, when the first treatment cycle with compound 0118 is completed and after allowing sufficient time for clearance of compound 0118 from the body of the patient, a radiolabeled analogue of compound 0118 is administered to the patient.

At 306, after a predetermined period of time to allow sufficient uptake of the radiolabeled analogue, the patient is scanned via a PET and/or SPECT imaging procedure.

At 308, it is determined whether the radiolabeled analogue is present in the image data.

If the radio labeled analogue is present in the image data, then at 310 continued use of compound 0118 is recommended.

In another embodiment, this decision is further refined. For example, this act may include determining an effectiveness of the treatment and basing the decision in part thereon. In this case, if the radio labeled analogue is present in the image data and the cancer has shrunk, use of compound 0118 is recommended. However, if the cancer has grown, continued use of compound 0118 may not be recommended.

If it is determined that the radio labeled analogue is not present in the image data, then at 312 a procedure that facilitates identifying whether the patient still has the cancer is performed.

At 314, based on a result of the procedure, it is determined whether the patient still has the cancer,

If the patient no longer has the cancer, then at 316 discontinued use of the compound is recommended.

If the patient still has the cancer, then at 318 compound 0118 is identified as no longer a treatment candidate.

It is to be appreciated that the ordering of the acts in the methods described herein is not limiting. As such, other orderings are contemplated herein. In addition, one or more acts may be omitted and/or one or more additional acts may be included.

Furthermore, one or more acts of the above methods may be implemented by way of computer readable instructions, encoded or embedded on computer readable storage medium, which, when executed by a computer processor(s), cause the processor(s) to carry out the described acts. Additionally or alternatively, at least one of the computer readable instructions is carried by a signal, carrier wave or other transitory medium.

Non-limiting examples of suitable radio labelled analogues of compound 0118 are discussed next.

The radiotracers described here are mimetics of compound 0118 and have been designed based on known structure-activity relationship (SAR) of a range of similar molecules, all characterized by the central calix[4]arene core, with hydrophobic substituents at the upper rim, and hydrophilic, basic substituents at the lower rim. Briefly, upper-rim substitution with linear and branched alkyl chains such as n-propyl, isobutyl, and tert-butyl groups leads to dramatic decrease in anti-angiogenic activity, strongly discouraging radio labeling strategies relying on introduction of radio labelled synthons at the upper rim. Although direct introduction of a single radionuclide at one of the aromatic rings of the calix[4]arene core may still be tolerated regarding steric constraints, this option is only viable for radionuclides, which can be introduced via directed electrophilic substitution, e.g. radiohalodestannylation reactions using the corresponding trialkylstannyl precursor. This approach may give access to radioiodinated and radiobrominated analogs of compound 0118, such as ¹²⁴I (t_{1/2}=4.2 days) for PET-, ¹²³I (t_{1/2}=13.2 hours) for SPECT-, and ⁷⁶Br (t_{1/2}=16.2 hours) for PET-imaging.

Unfortunately, widespread use of ⁷⁶Br is currently limited by its poor availability, while the rather long half-life of ¹²⁴I and the associated high radiation dose hampers widespread use of this radionuclide in the clinic. Clearly, ¹⁸F with its half-life of 110 min, comparably low positron energy, and excellent availability remains the PET-radionuclide of choice. Different from the other radiohalogens, however, direct introduction of ¹⁸F on one of the aromatic rings of compound 0118 is challenging, since the usually employed nucleophilic aromatic radiofluorination reaction only proceeds efficiently for activated, electron-poor aromatic systems. Since extended alkyl chains are precluded by the SAR, a single upper-rim [¹⁸F]fluoromethyl group, which is accessible via more facile aliphatic radio fluorination, may be considered as a final option. However, the stability of benzylfluorides is known to be limited, both in vitro and in vivo, particularly in case of electron-rich aromatic systems, rendering this approach less favourable.

As an alternative to upper-rim substitution, [¹⁸F]-labeled synthons may also be introduced at the lower rim or at an equatorial position of the calix[4]arene core of compound 0118. While the latter class of compounds is hitherto unknown, compound 0118 derivatives with different hydrophilic, basic substituents on the lower rim have been prepared and characterized in vitro and in vivo, indicating that certain variations of lower rim substituents are tolerable without complete loss of anti-angiogenic activity. Based on this knowledge, close mimetics of compound 0118 have been produced, where one of the four lower rim substituents is replaced by a substituent containing an alkyne- or azide-functional group, providing access to [¹⁸F]fluoroalkyltriazole-labeled compound 0118 analogues via Huisgen 1,3-dipolar cycloaddition reaction with the corresponding [¹⁸F]fluoroalkynes and [¹⁸F]fluoroazides, respectively. Most importantly, one particular representative of this class of compounds (compound 5) has been prepared and showed comparable anti-angiogenic potency in endothelial cell proliferation assays as compound 0118, confirming the validity of this approach. In addition, successful radio synthesis of an [¹⁸F]fluoroalkyltriazole compound 0118 analogue (compound [¹⁸F]5) has been demonstrated.

FIG. 4 shows the general structure of the compound 0118 and FIGS. 5, 6, 7, and 8 summarize the general structure of radiolabelled analogs of the compound 0118. As many as three different classes of radiolabelled analogs may be distinguished: FIG. 5 shows a Class 1 analog (including triazole regioisomers) where one of the lower rim substituents of the compound 0118 has been replaced by an [¹⁸F]fluoroalkyltriazole moiety. FIG. 6 shows a Class 2a analog modified with an equatorial [¹⁸F]fluoroalkyl chain. FIG. 7 shows a Class 2b analog (including triazole regioisomers) modified with an equatorial [¹⁸F]fluoroalkyltriazole moiety. Substituents R¹, R², R³, R⁴, and R⁵ in FIGS. 5, 6, and 7, could, for example, independently be chosen from alkyl, cycloalkyl, heterocycloalkyl, aralkyl, heteroaralkyl, alkoxy, thioalkoxy, cycloalkylalkoxy, or heterocycloalkylalkoxy, and each of these groups could include halogen, hydroxyl, sulfhydryl, amide, ester, (poly)ether, phosphonate, sulfonate, and/or keto functionalities. Preferably, however, R¹, R², R³, R⁴, and R⁵ are chosen from branched or linear alkyl- and/or (poly)-ether chains, such as polyethyleneglycol (PEG). FIG. 8 shows Class 3 upper-rim radiohalogenated analogues of the compound 0118, in particular, but not limited to radioiodinated and radiobrominated derivatives accessible via radiohalodestannylation from the corresponding tributylstannyl precursor. In general, substituent X in FIG. 8 denotes all radioactive isotopes of the halogens F, Cl, Br, I, At.

The following provides several non-limiting examples.

### EXAMPLE I

Synthesis of a compound 0118 analogue (compound 4) bearing a single alkyne functional group at the lower rim (precursor for an [¹⁸F]-labelled compound 0118 analogue belonging to the ‘Class 1’ radiotracers) and ‘Click’-reaction to form the corresponding [¹⁹F]-reference compound 5. FIG. 9 shows a synthetic strategy towards preparation of the alkyne-functionalized compound 0118 analogue (compound 4), [¹⁹F]-reference compound 5, and 2-azidoethyl-4-toluenesulfonate precursor 8. The first step is the selective alkylation of tetrahydroxycalix[4]arene with 4-bromobut-1-yne to afford monoalkylated calix[4]arene 2. Major complications in this step are the slow reaction rate and the concomitant formation of bis-alkylated by-products, which are difficult to remove. An optimized procedure using the more reactive 4-iodobut-1-yne instead of 4-bromobut-1-yne and sequential addition of multiple portions of sodium methoxide allowed preparation of a 3:1 mixture of compound 2 (26% isolated yield) and starting material 1. Since removal of the remaining starting material proved difficult, this mixture was directly used in the next step without further purification. Treatment with excess ethyl bromoacetate in the presence of potassium carbonate gave compound 3 in 67% yield after repeated purification by silica column chromatography, although the material still contained impurities. The partly purified material was reacted with N,N-dimethylethylenediamine and the crude material purified by prep. HPLC to obtain target compound 4 in a purity of >99%. Copper-catalyzed azide-alkyne cycloadition reaction (CuAAC) of crude 4 with freshly prepared 2-fluoroethylazide followed by prep. HPLC yielded reference compound 5 in a purity of >99%.

General methods. Unless stated otherwise, all reactions were performed in dried glassware under nitrogen. NMR spectra were recorded on either a Varian VNMRS spectrometer with a 7.05 Tesla magnet from Oxford Instruments and an indirect detection probe at 300 MHz (′H-NMR), or a Varian MP300 spectrometer with a 7.05 Tesla magnet from Oxford Instruments containing a 4 nuclei auto-switchable probe. Chemical shift values are reported in δ (ppm) referenced to the residual protic solvent peaks (CDCl₃: δ 7.26 for ¹H and δ 77.0 for ¹³C). ¹H-NMR multiplicities are abbreviated as follows: s=singlet, d=doublet, t=triplet, q=quartet, dd=doublet of doublets, dt=doublet of triplets, m=multiplet, br=broad. ¹³C-NMR multiplicities (q=quaternary, t=tertiary, s=secondary and p=primary) were distinguished using an attached proton test (APT). Preparative HPLC was performed on System A: Instrument: Agilent 1100 series with UV detector, equipped with a Gemini NX C₁₈ 100 A Axia (100×30 mm, 5 um) column. Flow: 40.0 mL/min. UV detection: 215, 254 nm; Mobile phase: linear gradient of 20 mM ammoniumbicarbonate in MilliQ (solvent A) and acetonitrile (solvent B). Gradient details: 50% B (0 min→3 min), 50% B→95% B (3 min→9 min), 95% B (9 min→10 min). Injection volume: 25 μL. Analytical HPLC-MS was carried out on two different systems. System B: Agilent 1100 series with UV detector and HP 1100 mass TOF detector, equipped with a Kinetex C18 (50×2.10 mm; 2.6 μm) column, variable wavelength UV-detector, and API ES TOF positive and negative mass detection. Column temperature: 35° C. Flow: 0.60 mL/min. Injection volume: 1 μL. Mobile phase: 9.65 g ammonium acetate, 2250 mL H₂O, 150 mL methanol, 100 mL acetonitrile (eluent A); 9.65 g ammonium acetate, 250 mL H₂O, 1350 mL methanol, 900 mL acetonitrile (eluent B). System C: Agilent 1100 series with UV detector and HP 1100 MSD mass detector, equipped with a Waters XBridge-C18 (50×4.6 mm, 3.5 μm) column. Column temperature: 22° C. Flow: 1.0 mL/min. Injection volume: 0.2 μL. Mobile phase: as described for System B. All reagents, including anhydrous solvents, were obtained from Sigma-Aldrich (St. Louis, Mo.) and Acros (Geel, Belgium), and used without further purification. 25,26,27,28-Tetrahydroxycalix[4]arene was purchased from Carbosynth Limited (Compton, UK). 2-Fluoroethyl-4-methylbenzenesulfonate was from Molekula (Gillingham, UK).

25,26,27-Trihydroxy-28-(3′-butynyloxy)calix[4]arene (2). To 25,26,27,28-tetrahydroxycalix[4]-arene 1 (4 g, 9.42 mmol) in acetonitrile (220 mL) was added freshly prepared NaOMe (600 mg, 11.12 mmol). The mixture was refluxed for 30 min, cooled and 4-iodobut-1-yne (3.29 g, 24 mmol) in acetonitrile (40 mL) was added. (4-Iodobut-1-yne was freshly prepared from NaI (7.2 g, 48 mmol) and 4-bromobut-1-yne (3.2 g, 24 mmol) in acetonitrile (40 mL) at reflux for 1 h.). The mixture was refluxed overnight and the conversion was checked by ¹H-NMR (24%). Additional NaOMe (400 mg, 7.41 mmol) was added and the mixture was refluxed over the weekend (conversion 32%). Additional NaOMe (400 mg, 7.41 mmol) was added and the mixture was stirred at reflux for another 48 h. NMR analysis showed a conversion of 39%. Then additional freshly prepared NaOMe (400 mg, 7.41 mmol) was added and the mixture was refluxed for another 48 h. Conversion (49%) and also 5% bis-alkylated material was formed. Then the mixture was worked up by evaporation of the solvent. Dichloromethane (100 mL) was added to the residue and the mixture was washed with water (3×50 mL). After evaporation of the organic solvents an off-white solid was isolated. The title compound was isolated as a mixture with unreacted 25, 26, 27, 28-tetrahydroxycalix[4]arene. The material was stirred in ethyl acetate (15 mL) and the solid was filtered. The mother liquor was evaporated and 1.5 g of crude product was isolated. According to ¹H-NMR this sample contains approx. 75% of the title compound (corresponding to 1.16 g, 2.43 mmol, 26% yield of 2) and 25% of starting material. The solid was used in the next step without further purification. ¹H-NMR (300 MHz, CDCl₃) δ=9.7 (s, 1H), 9.17 (s, 2H), 7.09 (m, 8H, ArH), 6.90 (m, 1H, ArH), 6.69 (m, 3H, ArH), 4.43 (d, 2H, J=13.0 Hz), 4.29 (m, 4H), 3.49 (d, 4H, J=12.9 Hz), 3.06 (dt, 2H, J₁=2.7 Hz, J₂=6.6 Hz), 2.23 (t, 1H, J=2.7 Hz). ¹³C-NMR (75 MHz, CDCl₃) 6=151.32 (q), 151.05 (q), 149.45 (q), 134.34 (q), 129.69 (q), 129.23 (q), 129.11 (q), 129.02 (q), 128.66 (q), 128.59 (t), 128.49 (t), 126.58 (q), 122.20 (t) 121.15 (t), 80.42 (q), 74.60 (t), 71.28 (s), 32.15 (s), 31.66 (s), 20.35 (s).

25,26,27-Tri[(ethoxycarbonyl)methoxy]-28-(3′-butynyloxy)calix[4]arene (3). To a solution of 25, 26, 27-trihydroxy-28-(3′-butynyloxy)calix[4]arene 2 (1.5 g with purity 75%, corresponding to 2.43 mmol of 2) in acetonitrile (20 mL) was added K₂CO₃ (964 mg, 6.98 mmol). The mixture was stirred for 30 min and then an excess of ethyl bromoacetate (2.63 g, 15.75 mmol) was added. The mixture was heated to 70° C. for 96 hrs. After cooling, the acetonitrile was evaporated. The residue was taken up in dichloromethane (100 mL). The organic layer was washed with water (2×50 mL). After separation the organic layer was evaporated. The impure compound was purified by column chromatography (silicagel, dichloromethane). Several impure fractions were isolated. The first combined fractions (900 mg) contained product and by-products. The second batch of combined fractions was enriched in product (1 g) and the third batch (500 mg) was a combination of fractions which contained mainly tetra ethyl ester. The first batch was purified by column chromatography again on silicagel with dichloromethane to remove by-products and then ethyl acetate:heptanes=1:2 to elute the product. Approximately 450 mg of a mixture was isolated which was enriched in product. This material was combined with the 1 g batch. A total of 1.45 g of material was purified again by silica column chromatography using a mixture of ethylacetate:heptanes=6:1 followed by ethylacetate:heptanes=4:1 to obtain the title compound (1.2 g, 1.63 mmol, 67%). Unfortunately, ¹H-NMR analysis still indicated presence of some impurities. A 300 mg batch was used in the next step without further purification. The remainder (900 mg) was again purified by repeated silica column chromatography, first using dichloromethane followed by ethyl acetate:heptanes=1:4, then toluene:ethyl acetate=95:5 to obtain the pure title compound (30 mg, 0.04 mmol) and a slightly less pure fraction (260 mg, 0.35 mmol). ¹H-NMR (300 MHz, CDCl₃) δ=6.79-6.68 (m, 6H, ArH), 6.57-6.53 (m, 6H, ArH), 4.85 (d, 2H J=13.6 Hz), 4.8 (s, 2H), 4.66 (d, 2H, J=13.5 Hz) 4.75-4.56 (4H), 4.28 (q, 4H, J=7.2 Hz), 4.24 (q, 2H, J=7.2 Hz), 4.1 (t, 2H, J=7.6 Hz), 3.24 (2×d, 4H, J=13.3 Hz), 2.92 (dt, 2H, J₁=2.6 Hz, J₂=7.6 Hz), 1.97 (t, 1H, J=2.6 Hz), 1.27-1.36 (m, 9H). ¹³C-NMR (75 MHz, CDCl₃) δ=170.41 (q), 170.11 (q), 156.59 (q), 156.34 (q), 155.53 (q), 135.62 (q), 135.10 (q), 134.61 (q), 134.25 (q), 128.93 (t), 128.77 (t), 128.68 (t), 128.57 (t), 123.07 (t), 122.93 (t), 82.19 (q), 72.74 (s), 71.79 (s), 71.70 (s), 69.36 (t), 60.96 (s), 60.79 (s), 31.48 (s), 31.29 (s), 20.03 (s), 14.50 (p), 14.44 (p). Analytical HPLC-MS was performed on System C (see general methods). Gradient: 20% B 95% B (0 min 1.5 min), 95% B (1.5 min 4.0 min). Retention time: 3.65 min. Purity>99.99% (UV, 215, 254 nm). MS calculated for C₄₄H₄₆O₁₀: 734.31; MS (API ES TOF Pos): m/z 752 ([M-NH₄]+, 757 ([M-Na]+).

25,26,27-Tris-N—(N,N-dimethyl-2-aminoethyl)carbamoylmethoxy-28-(3′-butynyloxy)calix[4]arene (4). To 25,26,27-tri[(ethoxycarbonyl)methoxy]-28-(3′-butynyloxy)-calix[4]arene 3 (300 mg, 0.41 mmol) was added under nitrogen N,N-dimethylethylenediamine (5 mL). The mixture was stirred at room temperature for 1 hr and was then stirred for 48 hrs at 50° C. The excess of N,N-dimethylethylenediamine was removed by evaporation under reduced pressure. A sample of 220 mg was dissolved in tetrahydrofuran (unstabilized, concentration of crude 70 mg/mL) and purified by preparative HPLC on system A (see general methods). Fractions containing the product (t_{R}=6.4 min; broad peak) were pooled and evaporated at the rotary evaporator to remove the acetonitrile. The water was removed by freeze-drying to obtain the pure target compound as an off-white foam (110 mg, 128 μmol, c.y. 38%). ¹H-NMR (300 MHz, CDCl₃) δ=6.74-6.67 (d, 6H, ArH), 6.6-6.47 (m, 6H, ArH), 4.57 (d, 2H J=13.9 Hz), 4.56-4.33 (m, 4H), 4.52 (s, 2H), 4.40 (d, 2H, J=14.1 Hz), 4.14 (t, 2H, J=7.3 Hz), 3.56-3.40 (m, 6H), 3.28 (d, 2H, 14.2 Hz), 3.26 (d, 2H, 13.8 Hz), 2.75 (dt, 2H, J=7.3 Hz, J=2.7 Hz), 2.53 (t, 4H, 6.6 Hz), 2.45 (t, 2H, 6.5 Hz), 2.26 (s, 12H), 2.2 (s, 6H), 2.09 (t, 1H, J=2.6 Hz). ¹³C-NMR (75 MHz, CDCl₃) δ=169.58 (q), 169.46 (q), 156.11 (q), 155.60 (q), 155.47 (q), 135.21 (q), 134.92 (q), 133.94 (q), 133.80 (q), 129.18 (t), 129.00 (t), 128.85 (t), 128.71 (t), 123.29 (t), 123.22 (t), 81.81 (q), 74.31 (s), 74.15 (s), 72.61 (s), 70.47 (t), 58.31 (s), 58.18 (s), 57.98 (s), 45.51 (p), 45.44 (p), 45.29 (p), 37.26 (s), 37.17 (s), 35.65 (s), 31.32 (s), 31.07 (s), 19.94 (s). Analytical HPLC-MS was performed on System B. Gradient: 20% B→90% B (0 min→1.0 min), 90% B→100% B (1.0 min→3.5 min), 100% B (3.5 min→4.0 min). Retention time: 2.03 min. Purity 99.64% (UV, 218 nm). MS calculated for C₅₀H₆₄N₆O₇: 860.48; MS (API ES TOF Pos): m/z 431 ([½M]+), 861 (M+), 883 ([M-Na]+). MS (API ES TOF Neg): m/z 859 (M−1), 919 ([M-CH₃COO]—).

2-Fluoroethylazide (6). To a solution of 2-fluoroethyl-4-methylbenzenesulfonate (640 mg, 2.93 mmol) in N,N-dimethylformamide (50 mL) was added sodium azide (570 mg, 8.8 mmol). The mixture was stirred at room temperature for 72 hrs. The reaction was followed by TLC (silicagel) heptanes: ethyl acetate=2:1. The reaction mixture was filtered and the filtrate containing the title compound was used without isolation for subsequent reactions. WARNING: Attempts to isolate neat 2-fluoroethyl azide may result in an explosion.

25,26,27-Tris-N—(N,N-dimethyl-2-aminoethyl)carbamoylmethoxy-28-{2′-[1-(2-fluorethyl)-1H-[1,2,3]triazo-4-yl]ethyloxy}calix[4]arene (5). To a stirred solution of Cu(II)SO₄.5H₂O (21.34 mg, 0.086 mmol) and (L)-ascorbic acid (30.29 mg, 0.172 mmol) in water (1 mL) under an N₂ atmosphere was added a solution of the crude 25,26,27-Tris-N—(N,N-dimethyl-2-aminoethyl)carbamoyloxymethoxy-28-(3′-butynyloxy)calix[4]arene 4 (50 mg, 0.057 mmol) in N,N-dimethylformamide (0.7 mL). After addition of 2-fluoroethyl azide (5.09 mg, 0.057 mmol) in N,N-dimethylformamide (1 mL) the mixture was stirred at room temperature overnight. The reaction mixture was evaporated to dryness. The solid residue was treated with dichloromethane (5 mL) and water (5 mL). A few drops of NaOH (1 M) were added. The layers were separated and the aqueous layer extracted again with dichloromethane (5 mL). The combined organic layers were washed with water (5 mL). After separation the organic layer was evaporated in vacuo. The crude material (40 mg) was taken up in tetrahydrofuran (unstabilized, 60 mg/mL, injection volume 25 μL) and purified by preparative HPLC on system A (see general methods). Fractions containing the product (t_{R}=6.8 min; broad peak) were pooled and evaporated at the rotary evaporator to remove the acetonitrile. The water was removed by freeze-drying. The target compound was isolated as a white solid 15 mg (15.7 μmol, c.y. 27.7%). A larger batch (50 mg) has been synthesized in the same way. ¹H-NMR (300 MHz, CDCl₃) δ=7.96 (m, 1H), 7.64 (m, 2H), 7.60 (s, 1H), 6.67 (s, 6H), 6.59-6.41 (m, 6H), 4.79 (dt, ²J_{HF}=46.9 Hz, ³J_{HH}=4.6 Hz, 2H), 4.74-4.48 (m, 8H), 4.27 (d, J=14.2 Hz, 4H), 4.26-4.18 (m, 2H), 3.55-3.33 (m, 6H), 3.28 (t, J=6.9 Hz, 2H), 3.23 (d, J=13.9 Hz, 4H), 2.51-2.40 (m, 6H), 2.22 (s, 6H), 2.20 (s, 12H). ¹³C-NMR (75 MHz, CDCl₃) δ=169.55 (q), 169.16 (q), 156.33 (q), 145.02 (q), 134.52 (q), 134.27 (q), 134.17 (q), 134.08 (q), 129.30 (t), 128.76 (t), 128.49 (t), 123.23 (t), 123.03 (t), 122.92 (t), 122.76 (t), 122.74 (t), 82.69 (s), 82.55 (q), 80.40 (s), 74.49 (s), 73.87 (s), 73.77 (s), 58.27 (s), 58.11 (s), 50.63 (s), 50.37 (s), 45.41 (p), 37.14 (s), 31.33 (s), 30.98 (s), 26.76 (s). ¹⁹F-NMR (CDCl₃) δ=−150.75-−151.23 (m, 1F). Analytical HPLC-MS was performed on System B (see general methods). Gradient: 20% B→95% B (0 min→1.5 min), 95% B (1.5 min→4.0 min). Retention time: 2.41 min. Purity 99.82% (UV, 224 nm). MS calculated for C₅₂H₆₈FN₉O₇: 949.52; MS (API ES TOF Pos): m/z 475 ([½M]+), 950 (M+), 977 ([M-Na]+).

2-Azidoethyl-4-methylbenzenesulfonate (8). To a solution of 2-azidoethanol 7 (2.61 g, 30 mmol) in dichloromethane (50 mL) at room temperature were consecutively added triethylamine (4.54 g, 45 mmol) and tosyl chloride (5.72 g, 30 mmol). The temperature initially dropped to 17° C. and then increased to 30° C. The solution was stirred at room temperature for 4 h. The mixture was washed with aqueous 1 N NaOH (2×50 mL). The organic layers were concentrated to a light yellow oil, 7 g. The product was purified by column chromatography (200 g SiO₂, heptane/ethyl acetate 4/1). Yield 5.5 g (22.8 mmol, 76%) of a colorless oil. ¹H-NMR (300 MHz, CDCl₃): δ=7.81 (d, 2H, J=8.5 Hz), 7.36 (d, 2H, J=8.5 Hz), 4.16 (t, 2H, J=5.0 Hz), 3.48 (t, 2H, J=5.0 Hz), 2.46 (s, 3H). Analytical HPLC-MS was performed on System C (see general methods). Gradient: 30% B (0 min→1 min), 30% B→100% B (1 min→5 min), 100% B (5 min→9 min). Purity 99.96% (UV 236 nm, 264 nm).

### EXAMPLE II

Synthesis of a compound 0118 analogue (compound 13) bearing a single equatorial alkyne substituent (precursor for an [¹⁸F]-labelled compound 0118 analogue belonging to the ‘Class 2b’ radiotracers) and ‘Click’-reaction to form the corresponding [¹⁹F]-reference compound 14. FIG. 10 shows a synthetic strategy towards preparation of precursor 13 and reference compound 14. Target compound 14 was prepared in a 6-step reaction sequence starting from tetrahydroxycalix[4]arene 1. Crucial step is the selective monoalkylation of tetramethoxycalix[4]arene 9 at an equatorial position of one of the methylene bridges to yield intermediate 10, which was achieved employing a similar procedure as described for alkylation of tetramethoxy-p-tert-butylcalix[4]arene. Although many different conditions were investigated, maximum conversion for the equatorial monoalkylation was about 80%, and removal of unreacted starting material by silica column chromatography led to substantial loss of product, reducing the isolated yield to 37% (72% before chromatography). Deprotection with boron tribromide in dichloromethane followed by reaction with excess ethyl bromoacetate in the presence of sodium carbonate gave tetraethyl ester 12 in 36% isolated yield over 2 steps. Refluxing with excess N,N-dimethylethylenediamine yielded ‘Click’-precursor 13, which was reacted with freshly prepared 2-fluoroethylazide in the presence of CuSO₄/ascorbic acid to obtain reference compound 14 in an overall yield of 8% from commercially available tetrahydroxycalix[4]arene.

General methods. Unless stated otherwise, all reactions were performed in dried glassware under nitrogen atmosphere. NMR spectra were recorded on a Bruker DPX300 spectrometer. Chemical shift values are reported in ppm relative to tetramethylsilane as the internal standard (TMS: 8=0 ppm for ¹H and 8=0 ppm for ¹³C). ¹H-NMR multiplicities are abbreviated as follows: s=singlet, d=doublet, t=triplet, q=quartet, dd=doublet of doublets, dt=doublet of triplets, bs=broad singlet. ¹³C-NMR multiplicities (q=quaternary, t=tertiary, s=secondary, and p=primary) were distinguished using a DEPT pulse sequence. Preparative column chromatography was performed on a Combiflash Companion apparatus (Teledyne Isco) employing pre-packed silica cartridges from Grace (Deerfield, Ill.). Preparative HPLC was performed using an Agilent 1200 apparatus, equipped with a C18 Zorbax column (21.2×150 mm, 5 μm particles) applying a linear gradient of acetonitrile (B) in water (A), both containing 0.1% TFA. Flow: 10 mL/min. UV detection: 215 nm, 254 nm. Gradient details: 30% B (0 min→5 min), 30% B 50% B (5 min→12 min), 50% B (12 min→15 min), 50% B 95% B (15 min→16 min), 95% B (16 min 18 min), 95% B 30% B (18 min→19 min), 30% B (19 min→22 min). Injection volume: 0.5 mL. All reagents, including anhydrous solvents, were obtained from Sigma-Aldrich (St. Louis, Mo.) and Acros (Geel, Belgium), and used without further purification. 25,26,27,28-Tetrahydroxycalix[4]arene was purchased from Carbosynth Limited (Compton, UK). 2-Fluoroethyl-4-methylbenzenesulfonate was from Molekula (Gillingham, UK).

25,26,27,28-Tetramethoxycalix[4]arene (9). 25,26,27,28-Tetrahydroxycalix[4]-arene 1 (7.5 g, 17.7 mmol) was dissolved in a 10:1 mixture of anhydrous tetrahydrofuran:N,N-dimethylformamide (165 mL). NaH (12 g of a 60% dispersion in mineral oil, 300 mmol) was triturated with n-pentane to remove the mineral oil. After drying under a gentle stream of nitrogen, the NaH was added to the precursor solution in tetrahydrofuran/N,N-dimethylformamide, followed by MeI (33 mL, 531 mmol). The reaction mixture was refluxed for 2 hours, treated with methanol (10 mL) to compose the excess of NaH, and evaporated in vacuo. The solid was partitioned between water (300 mL) and dichloromethane (300 mL). The layers were separated and the water layer was again extracted with dichloromethane (300 mL). The combined organic layers were back-extracted with water (150 mL), dried on MgSO₄, and evaporated in vacuo. Traces of MeI and N,N-dimethylformamide were efficiently removed by coevaporation with dichloromethane. Yield: 8.3 g (17.3 mmol, 98%) of a white to slightly yellow solid. ¹H-NMR (300 MHz, CD₃CN sat. with NaI) δ 7.34 (d, J=7.7 Hz, 8H), 6.92 (t, J=7.7 Hz, 4H), 4.28 (d, J=12.5 Hz, 4H), 4.14 (s, 12H), 3.60 (d, J=12.5 Hz, 4H). ¹³C-NMR (75 MHz, CD₃CN sat. with NaI) 6=153.80 (q), 136.33 (q) 130.26 (t), 126.77 (t), 65.58 (p), 29.75 (s).

25,26,27,28-Tetramethoxy-2-(prop-2′-yn-1′-yl)calix[4]arene (10). 25,26,27,28-Tetramethoxycalix[4]arene 9 (1 g, 2.08 mmol) was dissolved in anhydrous tetrahydrofuran (30 mL). The solution was cooled in an ice-bath and n-BuLi (7.8 mL of 1.6 M n-BuLi in hexanes, 12.5 mmol) was added dropwise The resulting dark-red solution was allowed to stir for 20 min and then added to a stirred, pre-cooled solution (ice-bath) of propargyl bromide (2.78 mL of an 80 wt. % solution in toluene, 25.0 mmol) in anhydrous tetrahydrofuran (15 mL). The reaction mixture was stirred for 1 h in an ice-bath, allowed to warm to room temperature (ca. 20 min), quenched with sat. aqueous KHSO₄ (10 mL), and evaporated in vacuo. The crude product was taken up in dichloromethane (200 mL) and water (100 mL). The organic extracts were washed with brine (100 mL), dried on MgSO₄, and filtered through a short column of silica to remove highly polar impurities (baseline spot on TLC). ¹H-NMR of the crude product (950 mg) indicated a conversion of ca. 80% (corresponding to 771 mg, 1.49 mmol, 72% yield of pure 10). The crude product was purified by column chromatography on silicagel employing a gradient of 0-10% of ethyl acetate in heptanes. Fractions containing the product were pooled, evaporated in vacuo, and co-evaporated with dichloromethane to remove traces of heptanes. Yield: 402 mg (0.78 mmol, 37%) of a white solid. ¹H-NMR (300 MHz, CD₃CN sat. with NaI) 6=7.43 (dd, J=7.9, 1.4 Hz, 2H), 7.35 (d, J=7.7 Hz, 6H), 6.97 (t, J=7.7 Hz, 2H), 6.92 (t, J=7.7 Hz, 2H), 4.95 (t, J=8.6 Hz, 1H), 4.28 (d, J=12.5 Hz, 2H), 4.27 (d, J=12.5 Hz, 1H), 4.18 (s, 6H), 4.14 (s, 6H), 3.62 (d, J=12.5 Hz, 2H), 3.60 (d, J=12.5 Hz, 1H), 3.15 (dd, J₁=8.4 Hz, J₂=2.5 Hz, 2H), 2.33 (t, J=2.5 Hz, 1H). ¹³C-NMR (75 MHz, CD₃CN sat. with NaI) 6=153.86 (q), 153.63 (q), 138.38 (q), 136.36 (q), 136.34 (q), 136.30 (q), 130.44 (t), 130.33 (t), 130.29 (t), 127.06 (t), 126.90 (t), 126.83 (t), 83.98 (q), 71.42 (t), 65.91 (p), 65.54 (p), 35.88 (t), 29.90 (s), 29.73 (s), 23.90 (s).

25,26,27,28-Tetrahydroxy-2-(prop-2′-yn-1′-yl)calix[4]arene (11). A stirred solution of 25,26,27,28-tetramethoxy-2-(prop-2′-yn-1′-yl)calix[4]arene 10 (379 mg, 0.73 mmol) in anhydrous dichloromethane (30 mL) was cooled to −78° C. After 20 min, a 1.0 M solution of BBr₃ in dichloromethane (4.75 mL, 4.75 mmol) was added dropwise, and the reaction mixture was kept at −78° C. for 1 h. The cooling bath was then removed, and stirring continued for 1 h. The reaction mixture was quenched with saturated aq. NaHCO₃ (120 mL), and additional dichloromethane (120 mL) was added. The organic layer was washed with water (120 mL), dried over MgSO₄, filtered, and evaporated in vacuo. The crude product (quantitative yield) was purified by silica column chromatography employing a gradient of 5-30% of ethyl acetate in heptanes to obtain the pure target compound as a white solid (36 mg, 0.078 mmol, 11%). ¹H-NMR (300 MHz, CDCl₃) δ=10.10 (s, 4H), 7.08-7.02 (m, 8H), 6.78 (t, J=7.7 Hz, 2H), 6.73 (t, J=7.7 Hz, 2H), 4.85 (t, J=7.5 Hz, 1H), 4.26 (d, J=13.9 Hz, 3H), 3.54 (d, J=13.9 Hz, 3H), 3.07 (d, J=7.5 Hz, 2H), 1.90 (t, J=2.4 Hz, 1H). ¹³C-NMR (75 MHz, CDCl₃) δ 149.98 (q), 148.96 (q), 130.29 (q), 129.17 (t), 129.09 (t), 129.03 (t), 128.44 (q), 128.33 (q), 128.28 (q), 124.54 (t), 122.61 (t), 122.41 (t), 82.45 (q), 69.56 (t), 35.47 (t), 31.94 (s), 31.84 (s), 22.32 (s).

25,26,27,28-Tetra[(ethoxycarbonyl)methoxy]-2-(prop-2′-yn-1′-yl)calix[4]arene (12). To a solution of 25,26,27,28-tetrahydroxy-2-(prop-2′-yn-1′-yl)calix[4]arene 11 (660 mg, ca. 1.4 mmol of crude 11 obtained after deprotection of pure 10) in dry acetonitrile (25 mL) was added finely powdered anhydrous Na₂CO₃ (1.45 g, 13.7 mmol). The resulting suspension was stirred for 1 h at 30° C., ethyl bromoacetate (1.52 mL, 13.7 mmol) was added, and the reaction mixture was refluxed for 24 hrs. After cooling down to room temperature, the salt was removed by filtration, and the filtrate concentrated in vacuo. The residue was taken up in dichloromethane (100 mL) and water (100 mL). The layers were separated, and the water layer was washed two times with dichloromethane (2×50 mL). The combined organic layers were dried over MgSO₄, filtered, and evaporated in vacuo. The crude was purified by chromatography column on silicagel using a gradient of 30-60% of ethyl acetate in heptanes to obtain the pure target compound as a pale yellow glassy solid (401 mg, 0.50 mmol, 36%). ¹H-NMR (300 MHz, CDCl₃) δ=6.70-6.58 (m, 12H), 5.36 (t, J=7.9 Hz, 1H), 4.95-4.72 (m, 11H), 4.25 (q, J=7.1 Hz, 4H), 4.20 (q, J=7.1 Hz, 4H), 3.25 (d, J=13.7 Hz, 1H), 3.23 (d, J=13.7 Hz, 2H), 2.82 (dd, J₁=7.9, J₂=2.6 Hz, 2H), 1.93 (t, J=2.6 Hz, 1H), 1.30 (t, J=7.1 Hz, 6H), 1.29 (t, J=7.1 Hz, 6H). ¹³C-NMR (75 MHz, CDCl₃) δ=170.20 (q), 170.05 (q), 155.93 (q), 155.79 (q), 136.66 (q), 134.65 (q), 134.64 (q), 134.59 (q), 128.98 (t), 128.55 (t), 128.41 (t), 124.88 (t), 122.98 (t), 122.84 (t), 83.60 (q), 71.73 (s), 71.26 (s), 69.72 (t), 60.56 (s), 60.50 (s), 36.04 (t), 31.64 (s), 31.38 (s), 23.67 (s), 14.24 (p), 14.19 (p).

25,26,27,28-Tetrakis-N—(N,N-dimethyl-2-aminoethyl)carbamoylmethoxy-2-(prop-2′-yn-1′-yl)calix[4]arene (13). To 25,26,27,28-Tetra[(ethoxycarbonyl)methoxy]-2-(prop-2′-yn-1′-yl)calix[4]arene (12) (351 mg, 0.435 mmol) under N₂ was added N,N-dimethylethylenediamine (7.5 mL) and the mixture was stirred at room temperature for 5 days. The excess of N,N-dimethylethylenediamine was evaporated in vacuo and remaining traces were removed by repeated co-evaporation with acetonitrile (2×50 mL). The crude material (430 mg) was triturated with diethyl ether (20 mL) and dried under high vacuum for several hours to yield the target compound as an off-white solid (369 mg, 0.378 mmol, 87%). According to ¹H-NMR, purity of this material was >90%. Part of the material (ca. 200 mg) was taken up in a mixture of acetonitrile and water (ca. 8 mL) and purified by preparative HPLC (see general methods). Fractions containing the product (broad peak; t_{R}=8.0-10.5 min) were pooled, evaporated at the rotary evaporator to remove the acetonitrile, and lyophilized to yield the TFA-salt of compound 13 as a pale yellow solid (170 mg, 119 μmol) The solid was taken up in dichloromethane (60 mL) and thoroughly extracted with saturated aq. NaHCO₃ (60 mL). The organic phase was dried over MgSO₄, filtered, and evaporated to obtain target compound 13 as the free tetra-amine. Yield: 68 mg (69.7 μmol) of a white solid. ¹H NMR (300 MHz, CDCl₃) δ=7.86 (bs, 2H), 7.64 (bs, 2H), 6.70-6.51 (m, 12H), 5.29 (t, J=7.6 Hz, 1H), 4.75 (d, J=14.3 Hz, 2H), 4.52 (d, J=14.2 Hz, 3H), 4.36 (d, J=14.3 Hz, 6H), 3.58-3.38 (m, 8H), 3.26 (dd, J=28.1, 14.1 Hz, 3H), 2.73 (dd, J₁=7.8 Hz, J₂=2.4 Hz, 2H), 2.55-2.46 (m, 8H), 2.23 (d, J=2.3 Hz, 24H), 1.96 (t, J=2.4 Hz, 1H). ¹³C-NMR (75 MHz, CDCl₃) δ=169.60 (q), 169.53 (q), 156.07 (q), 155.75 (q), 136.84 (q), 134.54 (q), 133.85 (q), 133.81 (q), 129.35 (t), 128.89 (t), 128.84 (t), 125.51 (t), 123.27 (t), 123.23 (t), 83.19 (q), 74.37 (s), 74.13 (s), 70.05 (t), 58.12 (s), 58.08 (s), 45.31 (p), 37.09 (s), 37.06 (s), 36.45 (t), 31.41 (s), 31.03 (s), 23.56 (s).

25,26,27,28-Tetrakis-N—(N,N-dimethyl-2-aminoethyl)carbamoylmethoxy-2-{[1-(2-fluorethyl)-1H-[1,2,3]triazo-4-yl]methyl}calix[4]arene (14). To a stirred solution of Cu(II)SO₄.5H₂O (67 mg, 0.268 mmol) and (L)-ascorbic acid (92 mg, 0.522 mmol) in water (3 mL) under an N₂ atmosphere was added a solution of the crude 25,26,27,28-tetrakis-N—(N,N-dimethyl-2-aminoethyl)carbamoylmethoxy-2-(prop-2′-yn-1′-yl)calix[4]arene (13) (169 mg, 0.173 mmol) in N,N-dimethylformamide (2.5 mL). A freshly prepared solution of 2-fluoroethyl azide (6) in N,N-dimethylformamide was added (3.6 mL of a 0.0588 M solution, 0.212 mmol), and the mixture was stirred at room temperature overnight. The reaction mixture was then evaporated in vacuo, and the residue was treated with dichloromethane (15 mL) and aqueous Na₂CO₃ (15 mL). The layers were separated and the aqueous layer was extracted again with dichloromethane (2×15 mL). The combined organic layers were dried over MgSO₄, filtered, and evaporated in vacuo to yield the target compound (133 mg, 0.125 mmol, 72%). The crude material was taken up in a mixture of acetonitrile and water (ca. 2.5 mL) and purified by preparative HPLC (see general methods). Fractions containing the product (broad peak; t_{R}=7.0-9.5 min) were pooled, partly evaporated at the rotary evaporator to remove the acetonitrile, and lyophilized to obtain the TFA-salt of compound 14 as a white solid (116 mg, 76 μmol, 44%). The solid was taken up in dichloromethane (30 mL) and thoroughly extracted with saturated aqueous NaHCO₃ (30 mL). The layers were separated and the aqueous layer was again extracted with dichloromethane (30 mL). The combined organic extracts were dried over MgSO₄, filtered, and evaporated to yield target compound 14 as the free tetra-amine. Yield: 48 mg (45 μmol, 26%) of a white solid. ¹H-NMR (300 MHz, CDCl₃) δ=8.10-7.60 (m, 4H), 7.69 (s, 1H), 6.70-6.52 (m, 12H), 5.51 (t, J=8.0 Hz, 1H), 4.77 (dt, ²J_{HF}=46.8 Hz, ³J_{HH}=4.6 Hz, 2H), 4.66-4.35 (m, 11H), 4.24-4.12 (m, 2H), 3.50-3.20 (m, 13H), 2.53-2.40 (m, 8H), 2.23 (s, 24H). ¹³C-NMR (75 MHz, CDCl₃) δ=169.60 (q), 169.56 (q), 155.95 (q), 155.74 (q), 146.81 (q), 137.16 (q), 134.45 (q), 133.90 (q), 129.03 (t), 128.96 (t), 128.76 (t), 125.89 (t), 123.27 (t), 123.21 (t), 82.70 (s), 80.42 (s), 74.13 (s), 73.95 (s), 58.16 (s), 58.00 (s), 50.57 (s), 50.30 (s), 45.34 (p), 45.23 (p), 37.05 (s), 36.97 (s), 36.33 (p), 31.31 (s), 31.09 (s), 30.18 (s).

### EXAMPLE III

Radiosynthesis of the [¹⁸F]-labelled compound 0118 analogue [¹⁸F]5 belonging to the ‘Class 1’ radiotracers. FIG. 11 shows a 2-step reaction sequence for radiosynthesis of [¹⁸F]5. The intermediate 2-[¹⁸F]fluoroethylazide was prepared by nucleophilic aliphatic radio fluorination of 2-azidoethyl-4-methylbenzenesulfonate and purified by co-distillation with acetonitrile. Copper-catalyzed azide-alkyne cycloaddition reaction (CuAAC) with calix[4]arene-alkyne precursor 4 then yielded radiotracer [¹⁸F]5, which was purified by preparative HPLC and formulated in dimethyl sulfoxide.

General methods. Radiosynthesis was performed on a custom-designed ‘Modular-Lab’ system. The individual modules, which make up this ‘Modular-Lab’ system are commercially available from Eckert & Ziegler Eurotope GmbH (Berlin, Germany), and are compliant with GMP, GLP, GAMP 5 and CFR21 Part 11 requirements. The heart of the system is formed by two Peltier reactor modules (PRM), which allow temperature control from −40° C. to +150° C. Both are equipped with magnetic stirrers, temperature and radioactivity sensors, pneumatic reactor lifts, and reactor cameras. The reactions were carried out in 3 mL borosilicate glass V-vials from Alltech (Grace Discovery Sciences, Deerfield, Ill.) equipped with reactor heads from Eckert & Ziegler and EPDM flat seals. Connections for liquid transfer were from FEP and PTFE tubing. Solvents were evaporated using a constant stream of argon regulated by a flow controller module. Radioactive exhaust vapors were condensed in a lead-shielded vacuum trap cooled with liquid nitrogen, and subsequently passed through an activated carbon filter. Liquid transfers were performed using either vacuum or a positive pressure of argon (1.5 bar), or both. The valve modules used for assembling the system were 3/2-way and 2/2-way solenoid valve modules (SMC-valves type LVM), a single stopcock module, and stopcock manifold modules for gas transfer and the final radiopharmaceutical formulation step. Semi-preparative HPLC purification was performed on a SymmetryPrep C18 column (100 Å, 7 μm, 7.8×300 mm, Waters Corporation, Milford, Mass.) integrated into an HPLC module equipped with an electrically driven 6-port-multi-channel valve, an Omron E3X-DA-S fluid sensor, a preparative sample loop, a radioactivity detector in series with a WellChrom fixed wavelength detector K-200 (λ=254 nm; Knauer GmbH, Berlin, Germany), and two WellChrom HPLC pumps K120 (Knauer GmbH). The complete system was placed in a hot-cell, whereas the PLC (programmable logic controller), the cooling unit, the cold trap and the vacuum pump were located in the service corridor behind the hot-cell. All processes were remotely controlled on a PC employing the dedicated Modular-Lab software interface from Eckert & Ziegler, which allows straightforward set-up of the interactive process panel, flexible programming, and provides GMP-compliant batch records including temperature, activity, and UV traces.

Analytical HPLC-analysis for monitoring reaction progress and composition of crude products and for quality control of the final tracer product was carried out on an Agilent 1100 Series system with a binary pump and a variable wavelength UV-detector (preset to 271 nm, which is λₘₐₓ of [¹⁹F]5) in series with a Gabi-Star radioactivity detector (Raytest GmbH, Straubenhardt, Germany). The samples were injected onto a Symmetry C18 column (100 Å, 5 μm, 3.9×150 mm, Waters Corporation, Milford, USA), which was eluted at 1 mL/min with a linear gradient of acetonitrile (B) in water (A), both containing 0.1% TFA. Two different gradients were used for analysis of the intermediate 2-[¹⁸F]fluoroethylazide (Gradient I: 3 min at 50% B followed by a linear gradient to 75% B in 7 min, a linear gradient to 95% B in 1 min, and subsequent isocratic elution for 2 min) and for analysis of the final [¹⁸F]-labelled compound 0118 analogue and the ‘Click’-reaction mixture (Gradient II: 3 min at 30% B followed by a linear gradient to 40% B in 5 min, isocratic elution for 2 min, a linear gradient to 95% B in 1 min, and subsequent isocratic elution for 2 min). Radioactivity and UV-retention times (t_{R}) for the starting materials, intermediates, and target compounds were as follows: 2-azidoethyl-4-methylbenzenesulfonate 8 (gradient 1: 5.50 min; gradient 2: 12.42 min); 2-[¹⁸F]fluoroethylazide [¹⁸F]6 (gradient 1: 2.87 min; gradient 2: 4.53 min); calix[4]arene-alkyne precursor 4 (gradient 2: 7.52 min); calix[4]arene-fluoroethyltriazole reference compound [¹⁹F]5 (gradient 2: 6.87 min); radiotracer [¹⁸F]5 (gradient 2: 7.23 min).

Radioactivity of the [¹⁸F]-charged QMA-cartridges, [¹⁸F]-intermediates, and the final product was measured in a calibrated digital ionization chamber (model VIK-202, Veenstra Instruments, Joure, The Netherlands). All reagents, including anhydrous solvents, were obtained from Sigma-Aldrich (St. Louis, Mo.) and Acros (Geel, Belgium), and used without further purification Anhydrous acetonitrile and Kryptofix 222 were from Merck (Darmstadt, Germany). Water was purified and de-ionized (18 MS2 cm) by means of a Milli-Q water filtration system (Millipore, Billerica, Mass.). The Sep-Pak® Plus Light C₈ cartridges for solid phase extraction were purchased from Waters (Milford, Mass.), the syringe filters (GD/X syringe filter, PTFE, 0.45 μm, with borosilicate prefilter) for filtration of the crude ‘Click’-reaction mixture were from Whatman (Kent, UK).

Radiosynthesis of [¹⁸F]5:[¹⁸F]F⁻ was purchased from BV Cyclotron VU (Amsterdam, The Netherlands). It was produced by the ¹⁸O (p,n)¹⁸F nuclear reaction in an IBA 18/9 cyclotron and subsequently trapped on a QMA-cartridge (Waters Sep-Pak® Plus Light QMA; carbonate form) for shipment. After arrival at our facilities (about 1-2 half-lives after EOB), [¹⁸F]F⁻ was eluted from the anion exchange column into a 3 mL V-vial using 1 mL of acetonitrile/water (9/1, v/v), which contained Kryptofix 222 (13 mg, 34 μmol) and K₂CO₃ (2 mg, 14 μmol). The solution was dried under an argon flow (˜70 mL/min) and reduced pressure at 70° C. for 5 min, 100° C. for 2 min, and 110° C. for 6 min (4 min lift-position ‘up’ and 2 min lift-position ‘down’). To remove residual water, anhydrous acetonitrile (0.9 mL) was added, and the solution was dried again at 70° C. for 2 min and 110° C. for 3 min (2 min lift-position ‘up’ and 1 min lift-position ‘down’). This co-evaporation cycle was repeated once.

After cooling to 40° C., 2-azidoethyl-4-methylbenzenesulfonate (5 μl) in acetonitrile (0.7 mL) was added to the K[¹⁸F]F-K222 residue, allowed to react for 15 min at 80° C., and the intermediate 2-[¹⁸F]fluoroethylazide was co-distilled at 90° C. under a constant stream of argon (20 mL/min) into a 3 mL V-vial containing calix[4]arene-alkyne precursor 4 (2.6 mg, 3.0 μmol) in DMSO (500 μL), which was pre-cooled to 30° C. After 10 min, the integrated radioactivity detectors in the reactors indicated that distillation was complete, and the radioactivity collected in the receiver vial was measured in a dose calibrator. Decay-corrected isolated radiochemical yield of 2-[¹⁸F]fluoroethylazide was 48±4% (n=2). HPLC-analysis of the distillate (without calix[4]arene-alkyne precursor 4 in the receiver vial) in previous experiments had indicated absence of 2-azidoethyl-4-methylbenzenesulfonate (starting material) in the UV-trace and a radiochemical purity of >99%. The volume of the 2-[¹⁸F]fluoroethylazide distillate in acetonitrile in these experiments was about 300-400 μL.

To the mixture of 2-[¹⁸F]fluoroethylazide and calix[4]arene-alkyne precursor 4 was then added the freshly prepared reagent mixture for the Huisgen cycloaddition reaction consisting of copper(II) sulphate pentahydrate (2.3 mg, 9.0 μmol) dissolved in water (150 μL) and (+)-sodium L-ascorbate (17.8 mg, 90 μmol) in 0.5 M sodium phosphate buffer pH 6 (100 μL). The mixture was heated to 80° C. for 15 min, diluted with water acidified with 0.1% TFA (1.5 mL), and a small sample was retrieved and immediately frozen in liquid nitrogen for later HPLC-analysis to determine the conversion in the ‘Click’-reaction (98%, n=2). The crude solution was passed through a Whatman GD/X syringe filter and loaded onto the C18 semi-preparative HPLC-column (pre-conditioned with 30% acetonitrile (eluant B) in water (eluant A), both acidified with 0.1% TFA) via a preparative sample loop. The flow was increased stepwise from 2 mL/min to 7 mL/min within 1 min, and elution with 30% B was continued for 3.5 min, followed by a linear gradient from 30% B to 40% B in 5 min, and subsequent isocratic elution with 40% B for 7 min. The retention time of [¹⁸F]5 was 9.1 min. The product fraction (total volume ca. 1.5-2 mL) was diverted into a septum-capped bottle containing water (35 mL). The purified radiotracer [¹⁸F]5 was trapped on a C₈ Sep-Pak® cartridge, rinsed with water (9 mL), eluted with ethanol (1 mL) into a 3 mL V-vial, evaporated to dryness at 80° C. under a stream of argon, and redissolved in dimethyl sulfoxide to the desired target concentration for subsequent in vitro and in vivo studies. Overall decay-corrected radiochemical yield of [¹⁸F]5 was 19.6±2.6% (n=2), and total synthesis time about 2 h. Radiochemical and chemical purity as assessed by HPLC-analysis were >99% and >93%, respectively (FIG. 12).

### EXAMPLE IV

Radiosynthesis of the [¹⁸F]-labelled compound 0118 analogue [¹⁸F]14 belonging to the ‘Class 2b’ radiotracers. [¹⁸F]14 was successfully prepared following essentially the same procedure as described for [¹⁸F]5 in example III, but using a linear gradient from 25% B to 35% B for prep. HPLC purification of the final tracer (all other parameters unchanged; retention time of [¹⁸F]14 was 8.9 min).

### EXAMPLE V

Synthesis of compound 0118 analogues bearing a single equatorial methyl-(compound 18a), n-propyl (compound 18b), or n-pentyl (compound 18c) substituent, which are specific examples of a new class of equatorially substituted 0118 analogues with general structure depicted in FIG. 13. The 5-step synthesis route for preparation of these compounds starting from tetrahydroxycalix[4]arene 1 is depicted in FIG. 14. For general methods, see example II.

General procedure for preparation of 25,26,27,28-tetramethoxy-2-alkylcalix[4]arenes (15a-c): 25,26,27,28-Tetramethoxycalix[4]arene (9) (2.00 g, 4.16 mmol) was dissolved in anhydrous THF (100 mL). The clear yellow solution was cooled to −20° C., and n-BuLi (11.7 mL of 1.6M n-BuLi in hexanes, 18.7 mmol) was added dropwise in the course of 30 min. The resulting blood-red solution was allowed to stir for 45 min. Alkyl iodide (37.4 mmol) was added, and the solution, which has turned orange-brown, was allowed to stir for 1 h while warming up to room temperature. After quenching with sat. aq. KHSO₄ (20 mL) and evaporation of most of the THF, water (80 mL) was added, and the product was extracted with dichloromethane (2×80 mL). The combined organic extracts were washed with brine (80 mL), dried on MgSO₄, and filtered over a short column of silica to remove highly polar impurities (baseline spot on TLC), followed by passing an additional 80 mL of dichloromethane, and evaporated in vacuo.

25,26,27,28-Tetramethoxy-2-methylcalix[4]arene (15a). Alkylation with methyl iodide (2.4 mL) yielded the title compound as an off-white solid (1.96 g, 3.96 mmol, 95%). ¹H-NMR (300 MHz, CD₃CN sat. with NaI) 6=7.41 (dd, ³J=7.9 Hz, ⁴J=1.5 Hz, 2H), 7.34 (d, ³J=7.7 Hz, 4H), 7.31 (dd, ³J=7.7 Hz, ⁴J=1.5 Hz, 2H), 6.96 (t, ³J=7.7 Hz, 2H), 6.92 (t, ³J=7.7 Hz, 2H), 4.88 (q, ³J=7.5 Hz, 1H), 4.28 (d, ²J=12.5 Hz, 2H), 4.27 (d, ²J=12.5 Hz, 2H), 4.14 (s, 6H), 4.12 (s, 6H), 3.60 (d, ²J=12.5 Hz, 3H), 1.71 (d, ³J=7.5 Hz, 3H). HRMS (ESI, m/z): Calculated for C₃₃H₃₄O₄H⁺ ([M-H]⁺): 495.2530. Found: 495.2553

25,26,27,28-Tetramethoxy-2-propylcalix[4]arene (15b). Alkylation with 1-iodopropane (3.7 mL) yielded the title compound as pale yellow solid (1.82 g, 3.48 mmol, 84%). ¹H-NMR (300 MHz, CD₃CN sat. with NaI) 6=7.40-7.28 (m, 8H), 6.99-6.88 (m, 4H), 4.69 (t, ³J=8.1 Hz, 1H), 4.27 (d, ³J=12.5 Hz, 3H), 4.14 (s, 6H), 4.12 (s, 6H), 3.60 (d, ²J=12.5 Hz, 2H), 3.59 (d, ²J=12.5 Hz, 1H), 2.20-2.02 (m, 2H), 1.44-1.24 (m, 2H), 0.99 (t, ³J=7.2 Hz, 3H). ¹³C-NMR (75 MHz, CD₃CN sat. with NaI) 6=153.89 (q), 153.79 (q), 140.00 (q), 136.41 (q), 136.37 (q), 136.16 (q), 130.29 (t), 129.89 (t), 126.99 (t), 126.79 (t), 65.68 (p), 65.56 (p), 36.88 (s), 36.04 (t), 29.92 (s), 29.77 (s), 22.60 (s), 14.60 (p). HRMS (ESI, m/z): Calculated for C₃₅H₃₈O₄H⁺ ([M-H]⁺): 523.2843. Found: 523.2818.

25,26,27,28-Tetramethoxy-2-pentylcalix[4]arene (15c). Alkylation with 1-iodopentane (4.9 mL) yielded the title compound as a white solid (1.91 g, 3.47 mmol, 83%). ¹H-NMR (300 MHz, CD₃CN sat. with NaI) 6=7.39-7.28 (m, 8H), 6.99-6.88 (m, 4H), 4.67 (t, ³J=8.1 Hz, 1H), 4.27 (d, ²J=12.5 Hz, 3H), 4.14 (s, 6H), 4.11 (s, 6H), 3.60 (d, ²J=12.5 Hz, 2H), 3.59 (d, ²J=12.5 Hz, 1H), 2.20-2.02 (m, 2H), 1.43-1.23 (m, 6H), 0.86 (t, ³J=7.2, Hz, 3H). ¹³C-NMR (75 MHz, CD₃CN sat. with NaI) δ=153.87 (q), 153.79 (q), 140.00 (q), 136.40 (q), 136.35 (q), 136.15 (q), 130.28 (t), 129.89 (t), 126.97 (t), 126.78 (t), 65.66 (p), 65.55 (p), 36.20 (t), 34.51 (s), 32.46 (s), 29.91 (s), 29.75 (s), 29.08 (s), 22.97 (s), 14.26 (p). HRMS (ESI, m/z): Calculated for C₃₇H₄₂O₄H⁺ ([M-H]⁺): 551.3156. Found: 551.3176.

General procedure for preparation of 25,26,27,28-tetrahydroxy-2-alkylcalix[4]arenes (16a-c): 25,26,27,28-Tetramethoxy-2-alkylcalix[4]arene (15a-c) (1.3-2.9 mmol) was dissolved in anhydrous dichloromethane (50 mL) and cooled to −78° C. A 1.0M solution of BBr₃ in dichloromethane (8.5-18.7 mL, 6.5 equiv.) was added dropwise via a syringe, and the resulting mixture was stirred for 1 h at −78° C., then allowed to warm to room temperature. After 30 min at room temperature, the reaction mixture was quenched by addition of saturated aq. NaHCO₃ (50 mL). Layers were separated, and the organic layer was washed with water (50 mL). After drying on MgSO₄, the extracts were passed over a thin column of silica, followed by rinsing with dichloromethane (200 mL), and evaporated in vacuo.

25,26,27,28-Tetrahydroxy-2-methylcalix[4]arene (16a). Demethylation of 25,26,27,28-tetramethoxy-2-methylcalix[4]arene (15a) (1.16 g, 2.35 mmol) with 1.0M BBr₃ in dichloromethane (15.3 mL, 15.3 mmol) yielded the title compound as a white foam (0.776 g, 1.77 mmol, 75%). ¹H-NMR (300 MHz, CDCl₃) δ=10.15 (s, 4H), 7.18-6.98 (m, 8H), 6.77 (t, ³J=7.5 Hz, 2H), 6.72 (t, ³J=7.7 Hz, 2H), 4.74 (q, ³J=7.2 Hz, 1H), 4.26 (d, ²J=13.9 Hz, 1H), 4.25 (d, ²J=13.9 Hz, 2H), 3.53 (d, ²J=13.9 Hz, 3H), 1.71 (d, ³J=7.2 Hz, 3H). HRMS (ESI, m/z): Calculated for C₂₉H₂₆O₄H⁺ ([M-H]⁺): 439.1904. Found: 439.1895.

25,26,27,28-Tetrahydroxy-2-propylcalix[4]arene (16b). Demethylation of 25,26,27,28-tetramethoxy-2-propylcalix[4]arene (15b) (1.50 g, 2.87 mmol) with 1.0M BBr₃ in dichloromethane (18.7 mL, 18.7 mmol) yielded the title compound as a white foam (1.28 g, 2.74 mmol, 95%). ¹H-NMR (300 MHz, CDCl₃) δ=10.12 (s, 4H), 7.19-6.88 (m, 8H), 6.80-6.64 (m, 4H), 4.53 (t, ³J=7.8 Hz, 1H), 4.26 (d, ²J=13.9 Hz, 1H), 4.25 (d, ²J=13.8 Hz, 2H), 3.53 (d, ²J=13.9 Hz, 3H), 2.22-2.05 (m, 2H), 1.42-1.23 (m, 2H), 0.95 (t, ³J=7.3 Hz, 3H). HRMS (ESI, m/z): Calculated for C₃₁H₃₀O₄H⁺ ([M-H]⁺): 467.2217. Found: 467.2214.

25,26,27,28-Tetrahydroxy-2-pentylcalix[4]arene (16c). Demethylation of 25,26,27,28-tetramethoxy-2-pentylcalix[4]arene (15c) (720 mg, 1.31 mmol) with 1.0M BBr₃ in dichloromethane (8.5 mL, 8.5 mmol) yielded the title compound as a white foam (594 mg, 1.20 mmol, 92%). ¹H-NMR (300 MHz, CDCl₃) δ=10.11 (s, 4H), 7.15-6.95 (m, 8H), 6.80-6.65 (m, 4H), 4.49 (t, ³J=7.7 Hz, 1H), 4.26 (d, ²J=13.9 Hz, 1H), 4.25 (d, ²J=13.9 Hz, 2H), 3.53 (d, ²J=13.9 Hz, 3H), 2.22-2.08 (m, 2H), 1.40-1.25 (m, 6H), 0.92-0.83 (m, 3H). HRMS (ESI, m/z): Calculated for C₃₃H₃₄O₄H⁺ ([M-H]⁺): 495.2530. Found: 495.2508.

General procedure for preparation of 25,26,27,28-tetra[(ethoxycarbonyl)methoxy]-2-alkylcalix[4]arenes (17a-c): 25,26,27,28-Tetrahydroxy-2-alkylcalix[4]arene (16a-c) (0.49-1.61 mmol) was dissolved in dry acetonitrile (15 mL) followed by addition of finely powdered anhydrous Na₂CO₃ (4.9-16.1 mmol, ca. 10 equiv.). The resulting suspension was stirred for 1 h at 30° C., ethyl bromoacetate (4.9-16.1 mmol, 10 equiv.) was added, and the reaction mixture was refluxed for 20 h. The solvent was evaporated in vacuo and the residue partitioned between dichloromethane (30 mL) and water (30 mL). The water layer was extracted with dichloromethane (2×15 mL). The combined organic extracts were dried on MgSO₄, filtered, and evaporated in vacuo to yield the crude product, which was purified by silica column chromatography employing a gradient of 15-30% of ethyl acetate in heptane to obtain the pure target compound.

25,26,27,28-Tetra[(ethoxycarbonyl)methoxy]-2-methylcalix[4]arene (17a). Precursor and Reagents: 25,26,27,28-Tetrahydroxy-2-methylcalix[4]arene (16a) (367 mg, 0.84 mmol); Na₂CO₃ (887 mg, 8.4 mmol); ethyl bromoacetate (0.93 mL, 8.4 mmol). Crude yield: yellow oil (536 mg, 0.68 mmol, 81%). Yield after silica column chromatography: white solid (155 mg, 0.20 mmol, 24%). ¹H-NMR (300 MHz, CDCl₃) δ=6.68-6.57 (m, 12H), 5.25 (q, ³J=7.2 Hz, 1H), 4.91 (d, ²J=13.5 Hz, 1H), 4.83 (d, ²J=13.7 Hz, 2H), 4.76-4.72 (m, 8H), 4.22 (q, ³J=7.2 Hz, 4H), 4.21 (q, ³J=7.2 Hz, 4H), 3.23 (d, ²J=13.7 Hz, 3H), 1.54 (d, ³J=7.2 Hz, 3H), 1.30 (t, ³J=7.2 Hz, 6H), 1.29 (t, ³J=7.2 Hz, 6H). ¹³C-NMR (75 MHz, CDCl₃) δ=170.19 (q), 170.07 (q), 155.86 (q), 155.53 (q), 139.57 (q), 134.85 (q), 134.56 (q), 134.23 (q), 128.49 (t), 128.43 (t), 125.03 (t), 122.95 (t), 122.82 (t), 71.52 (s), 71.31 (s), 60.54 (s), 60.48 (s), 31.51 (s), 31.44 (s), 30.97 (t), 20.45 (p), 14.21 (p). HRMS (ESI, m/z): Calculated for C₄₅H₅₀O₁₂H⁺ ([M-H]⁺): 783.3375. Found: 783.3378.

25,26,27,28-Tetra[(ethoxycarbonyl)methoxy]-2-propylcalix[4]arene (17b). Precursor and Reagents: 25,26,27,28-Tetrahydroxy-2-propylcalix[4]arene (16b) (228 mg, 0.49 mmol); Na₂CO₃ (518 mg, 4.9 mmol); ethyl bromoacetate (0.54 mL, 4.9 mmol). Crude yield: yellow oil (373 mg, 0.46 mmol, 94%). Yield after silica column chromatography: white solid (122 mg, 0.15 mmol, 31%). ¹H-NMR (300 MHz, CDCl₃) δ=6.67-6.55 (m, 12H), 5.02 (t, ³J=7.7 Hz, 1H), 4.89 (d, ²J=13.5 Hz, 2H), 4.86 (d, ²J=13.7 Hz, 1H), 4.85-4.68 (m, 8H), 4.23 (q, ³J=7.2 Hz, 4H), 4.20 (q, ³J=7.2 Hz, 4H), 3.24 (d, ²J=13.7 Hz, 1H), 3.22 (d, ²J=13.5 Hz, 2H), 1.88 (q, ³J=7.3 Hz, 2H), 1.48 (sext, ³J=7.3 Hz, 2H), 1.30 (t, ³J=7.2 Hz, 6H), 1.28 (t, ³J=7.2 Hz, 6H), 0.98 (t, ³J=7.3 Hz, 3H). ¹³C-NMR (75 MHz, CDCl₃) δ=170.24 (q), 169.97 (q), 155.93 (q), 155.73 (q), 138.22 (q), 134.75 (q), 134.61 (q), 134.50 (q), 128.48 (t), 128.43 (t), 128.31 (t), 125.29 (t), 122.94 (t), 122.77 (t), 71.50 (s), 71.15 (s), 60.49 (s), 60.44 (s), 36.63 (s), 36.39 (t), 31.69 (s), 31.39 (s), 21.47 (s), 14.48 (p), 14.24 (p), 14.19 (p). HRMS (ESI, m/z): Calculated for C₄₇H₅₄O₁₂H⁺ ([M-H]⁺): 811.3688. Found: 811.3675.

25,26,27,28-Tetra[(ethoxycarbonyl)methoxy]-2-pentylcalix[4]arene (17c). Precursor and Reagents: 25,26,27,28-Tetrahydroxy-2-pentylcalix[4]arene (16c) (500 mg, 1.01 mmol); Na₂CO₃ (1.07 g, 10.1 mmol); ethyl bromoacetate (1.12 mL, 10.1 mmol). Crude yield: yellow oil (472 mg, 0.56 mmol, 56%). Yield after silica column chromatography: white solid (138 mg, 0.16 mmol, 16%). ¹H-NMR (300 MHz, CDCl₃) δ=6.70-6.55 (m, 12H), 4.99 (t, ³J=7.7 Hz, 1H), 4.90 (d, ²J=13.5 Hz, 2H), 4.85 (d, ²J=13.8 Hz, 1H), 4.85-4.68 (m, 8H), 4.23 (q, ³J=7.2 Hz, 4H), 4.19 (q, ³J=7.2 Hz, 4H), 3.24 (d, ²J=13.8 Hz, 1H), 3.22 (d, ²J=13.5 Hz, 2H), 1.88 (q, ³J=7.3 Hz, 2H), 1.51-1.38 (m, 2H), 1.38-1.26 (m, 4H), 1.30 (t, ³J=7.2 Hz, 6H), 1.28 (t, ³J=7.2 Hz, 6H), 0.88 (t, ³J=7.1 Hz, 3H). ¹³C-NMR (75 MHz, CDCl₃) δ=170.27 (q), 169.98 (q), 155.96 (q), 155.73 (q), 138.20 (q), 134.72 (q), 134.63 (q), 134.54 (q), 128.56 (t), 128.48 (t), 128.43 (t), 128.29 (t), 125.29 (t), 122.95 (t), 122.77 (t), 71.52 (s), 71.14 (s), 60.50 (s), 60.45 (s), 36.66 (t), 34.33 (s), 32.29 (s), 31.69 (s), 31.39 (s), 28.04 (s), 22.63 (s), 14.24 (p), 14.20 (p), 14.10 (p). HRMS (ESI, m/z): Calculated for C₄₉H₅₈O₁₂Na⁺ ([M-Na]⁺): 861.3820. Found: 861.3856.

General procedure for preparation of 25,26,27,28-tetrakis-N—(N,N-dimethyl-2-aminoethyl)carbamoylmethoxy-2-alkylcalix[4]arenes (18a-c): A solution of 25,26,27,28-tetra[(ethoxycarbonyl)methoxy]-2-alkylcalix[4]arene (17a-c) (0.22-0.60 mmol) in N,N-dimethylethylenediamine (10 mL) was stirred at 50° C. for 48 hrs. The excess of N,N-dimethylethylenediamine was evaporated under reduced pressure and the resulting crystals were triturated twice with diethyl ether (first 10 mL, then 5 mL) and dried in vacuo. The crude product was then dissolved in 0.2M aq. HCl, and purified by preparative HPLC (see general methods). Fractions containing the product were pooled, evaporated at the rotary evaporator to remove the acetonitrile, and lyophilized to yield the TFA-salt of the pure product. To obtain the free tetra-amine, the salt was taken up in saturated aq. NaHCO₃ (30 mL) and extracted with dichloromethane (2×30 mL). The combined organic extracts were dried on MgSO₄, filtered, and evaporated in vacuo.

25,26,27,28-Tetrakis-N—(N,N-dimethyl-2-aminoethyl)carbamoylmethoxy-2-methylcalix[4]arene (18a). Precursor: 25,26,27,28-tetra[(ethoxycarbonyl)methoxy]-2-methylcalix[4]arene (17a) (176 mg, 224 μmol). Crude yield: off-white solid (170 mg, 179 μmol, 80%). Preparative HPLC: t_{R} (product)=8.3-10.8 min (broad peak). Yield after preparative HPLC (TFA-salt): white solid (107 mg, 76 μmol, 34%). Yield after extraction (free tetra-amine): white solid (53 mg, 56 μmol, 25%). ¹H-NMR (300 MHz, CDCl₃) δ=7.75 (bs, 2H), 7.61 (bs, 2H), 6.70-6.50 (m, 12H), 4.99 (q, ³J=7.2 Hz, 1H), 4.63 (d, ³J=14.1 Hz, 2H), 4.53-4.32 (m, 9H), 3.53-3.35 (m, 8H), 3.27 (d, ²J=14.1 Hz, 2H), 3.23 (d, ²J=12.8 Hz, 1H), 2.50-2.41 (m, 8H), 2.20 (bs, 24H), 1.49 (d, ³J=7.2 Hz, 3H). ¹³C-NMR (75 MHz, CDCl₃) δ=169.55 (q), 169.53 (q), 155.75 (q), 155.66 (q), 139.48 (q), 134.43 (q), 134.15 (q), 133.83 (q), 128.88 (t), 128.82 (t), 128.76 (t), 125.37 (t), 123.29 (t), 123.22 (t), 74.36 (s), 74.09 (s), 58.15 (s), 58.13 (s), 45.36 (p), 45.34 (p), 37.14 (s), 37.09 (s), 31.45 (t), 31.21 (s), 31.09 (s), 20.31 (p). HRMS (ESI, m/z): Calculated for C₅₃H₇₄N₈O₈H⁺ ([M-H]⁺): 951.5702. Found: 951.5727.

25,26,27,28-Tetrakis-N—(N,N-dimethyl-2-aminoethyl)carbamoylmethoxy-2-propylcalix[4]arene (18b). Precursor: 25,26,27,28-tetra[(ethoxycarbonyl)methoxy]-2-propylcalix[4]arene (17b) (486 mg, 599 μmol). Crude yield: off-white solid (584 mg, 596 μmol, 99%). Preparative HPLC: t_{R} (product)=10.8-12.3 min. Yield after preparative HPLC (TFA-salt): white solid (343 mg, 239 μmol, 40%). Yield after extraction (free tetra-amine): white solid (158 mg, 161 μmol, 27%). ¹H-NMR (300 MHz, CDCl₃) δ=7.77 (bs, 2H), 7.64 (bs, 2H), 7.71-6.50 (m, 12H), 4.88 (t, ³J=7.7 Hz, 1H), 4.64 (d, ²J=14.1 Hz, 2H), 4.57-4.32 (m, 9H), 3.52-3.36 (m, 8H), 3.28 (d, ²J=14.1 Hz, 2H), 3.22 (d, ²J=14.1 Hz, 1H), 2.50-2.41 (m, 8H), 2.21 (s, 12H), 2.20 (s, 12H), 1.91-1.77 (m, 2H), 1.46 (sext, ³J=7.2 Hz, 2H), 0.97 (t, ³J=7.2 Hz, 3H). ¹³C-NMR (75 MHz, CDCl₃) δ=169.55 (q), 169.52 (q), 155.95 (q), 155.62 (q), 138.27 (q), 134.51 (q), 133.95 (q), 133.80 (q), 128.89 (t), 128.79 (t), 128.68 (t), 125.74 (t), 123.19 (t), 74.22 (s), 74.01 (s), 58.13 (s), 45.33 (p), 37.12 (s), 37.06 (s), 36.90 (t), 36.45 (s), 31.33 (s), 31.15 (s), 21.60 (s), 14.46 (p). HRMS (ESI, m/z): Calculated for C₅₅H₇₈N₈O₈H⁺ ([M-H]⁺): 979.6015. Found: 979.6026.

25,26,27,28-Tetrakis-N—(N,N-dimethyl-2-aminoethyl)carbamoylmethoxy-2-pentylcalix[4]arene (18c). Precursor: 25,26,27,28-tetra[(ethoxycarbonyl)methoxy]-2-pentylcalix[4]arene (17c) (268 mg, 319 μmol). Crude yield: off-white solid (313 mg, 311 μmol, 97%). Preparative HPLC: t_{R} (product)=12.8-14.0 min. Yield after preparative HPLC (TFA-salt): white solid (177 mg, 121 μmol, 38%). Yield after extraction (free tetra-amine): white solid (110 mg, 109 μmol, 34%). ¹H-NMR (300 MHz, CDCl₃) δ=7.77 (bs, 2H), 7.63 (bs, 2H), 6.70-6.48 (m, 12H), 4.87 (t, ³J=7.5 Hz, 1H), 4.65 (d, ²J=14.1 Hz, 2H), 4.56-4.30 (m, 9H), 3.52-3.37 (m, 8H), 3.28 (d, ²J=14.1 Hz, 2H), 3.22 (d, ²J=14.1 Hz, 1H), 2.52-2.40 (m, 8H), 2.22 (s, 12H), 2.21 (s, 12H), 1.91-1.77 (m, 2H), 1.51-1.36 (m, 2H), 1.36-1.23 (m, 4H), 0.88 (t, ³J=7.1 Hz, 3H). ¹³C-NMR (75 MHz, CDCl₃) δ=169.55 (q), 169.50 (q), 155.97 (q), 155.61 (q), 138.30 (q), 134.51 (q), 133.94 (q), 133.80 (q), 128.88 (t), 128.79 (t), 128.68 (t), 125.72 (t), 123.21 (t), 123.18 (t), 74.24 (s), 74.00 (s), 58.14 (s), 45.33 (p), 45.31 (p), 37.24 (t), 37.14 (s), 37.05 (s), 34.26 (s), 32.30 (s), 31.34 (s), 31.14 (s), 28.35 (s), 22.73 (s), 14.15 (p). HRMS (ESI, m/z): Calculated for C₅₇H₈₂N₈O₈H′ ([M-H]⁺): 1007.6328. Found: 1007.6350.

### EXAMPLE VI

Evaluation of the anti-angiogenic activity of lower-rim substituted 0118 analogues (compound 4, compound 5) and equatorially substituted 0118 analogues (compound 13, compound 14, compound 18a, compound 18b, compound 18c). FIG. 15 shows the inhibitory effect of these novel 0118 analogues on proliferation of MA148 human ovarian carcinoma cells using a [³H]thymidine incorporation assay as readout (Dings et al., J Natl Cancer Inst 2006, 98(13), 932-936). Briefly, FIG. 15 shows that all of the equatorially substituted 0118 analogues more potently inhibit MA148 cell proliferation than parent compound 0118. In particular, compound 13, compound 14, and compound 18b, appear to be at least three times more potent than 0118, causing more than 70% inhibition of cell proliferation at a concentration of 0.5 μM. Also the lower-rim substituted analogue compound 5 appears to be a more potent inhibitor of MA148 cell proliferation, while compound 4 is at least equipotent.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A method for determining whether compound 0118 is a candidate treatment for a patient, comprising:
processing, via a processor, image data of tissue of interest of a patient including a cancer to determine whether a radiolabeled analog of compound 0118 is present in the tissue of interest represented in the image data; and
generating a signal indicating that compound 0118 is a candidate treatment for the patient in response to the determining that the radiolabeled analog of compound 0118 is present in a predetermined amount in the tissue of interest represented in the image data,
wherein the presence of the radiolabeled analog of compound 0118 in the tissue of interest indicates presence of a sub-type of cancer having a galectin-1 molecular target, which is a sub-type treatable by compound 0118.

2. The method of claim 1, further comprising:
visually presenting the image data and a notification indicating that compound 0118 is a candidate treatment for the patient.

3. The method of claim 1, wherein the signal indicates that compound 0118 is not a candidate treatment in response to determining that the radiolabeled analog of compound 0118 is not present in the predetermined amount in the tissue of interest represented in the image data.

4. The method of claim 1, further comprising:
determining the patient has cancer based on image data from an initial scan of the patient which was performed prior to all of the acts of claim 1.

5. The method of claim 4, wherein the initial scan includes one or more of a [¹⁸F]Fluorodeoxyglucose, [¹⁸F]fluoride, [¹⁸F]deoxyfluorothymidine, [¹⁸F]fluoromisonidazole, [¹¹C]choline, or [¹¹C]methionine based PET scan.

6. The method of claim 1, further comprising:
processing image data of a subsequent scan performed after at least one treatment with compound 0118 to determine whether the radiolabeled analog of compound 0118 is present in the tissue of interest represented in the image data; and
generating and presenting a recommendation signal recommending ceasing treatment with compound 0118 in response to the image data of the subsequent scan indicating the radiolabeled analog of compound 0118 is not present in the predetermined amount in the tissue of interest represented in the image data.

7. The method of claim 6, further comprising:
determining an absence of the cancer from the tissue of interest based on image data from a second subsequent scan of the tissue of interest; and
generating and presenting a notification indicating the cancer is no longer present in the tissue of interest.

8. The method of claim 6, further comprising:
determining a presence of the cancer from the tissue of interest based on a second subsequent scan of the tissue of interest; and
generating and presenting a notification indicating compound 0118 is no longer effective to treat the cancer.

9. The method of claim 1, further comprising:
processing image data of a subsequent scan of the patient after at least one treatment with compound 0118 to determine whether the radiolabeled analog of compound 0118 is present in the tissue of interest represented in the image data; and
generating and presenting a recommendation signal recommending continuing treatment with compound 0118 in response to the image data of the subsequent scan indicating the radiolabeled analog of compound 0118 is present in the predetermined amount in the tissue of interest represented in the image data.

10. The method of claim 9, further comprising:
comparing the image data of the scan and the image data of the subsequent scan;
determining a change in geometry of the cancer based on the comparison; and
presenting a notification indicating the change in geometry.

11. A method for monitoring treatment of cancer with compound 0118, comprising:
processing image data of a treatment scan performed after at least one treatment with compound 0118 to determine whether a radiolabeled analog of compound 0118 is present in tissue of interest represented in the image data, wherein the presence of the radiolabeled analog of compound 0118 in the tissue of interest indicates presence of a sub-type of cancer having a galectin-1 molecular target, which is a sub-type of treatable by compound 0118; and
generating and presenting a first recommendation signal recommending continuing treatment with compound 0118 in response to the image data of the treatment scan indicating the radiolabeled analog of compound 0118 is present in a predetermined amount in the tissue of interest represented in the image data.

12. The method of claim 11, further comprising:
comparing the image data of the treatment scan and the image data of an earlier scan;
determining a change in geometry of the cancer based on the comparison; and
presenting a notification indicating the change in geometry.

13. The method of claim 11, further comprising:
generating and presenting a second recommendation signal recommending ceasing treatment with compound 0118 in response to the image data of the treatment scan indicating the radiolabeled analog of compound 0118 is not present in the predetermined amount in the tissue of interest represented in the image data.

14. The method of claim 13, further comprising:
determining an absence of the cancer from the tissue of interest based on a subsequent scan of the tissue of interest therefrom; and
generating and presenting a notification indicating the cancer is no longer present in the tissue of interest.

15. The method of claim 13, further comprising:
determining a presence of the cancer from the tissue of interest based on a subsequent scan of the tissue of interest; and
generating and presenting a notification indicating compound 0118 is no longer effective to treat the cancer.

16. The method of claim 11, further comprising:
determining the patient has cancer based on image data from an initial scan performed prior to all of the acts of claim 11.

17. The method of claim 16, wherein the initial scan includes one or more of a [¹⁸F]Fluorodeoxyglucose, [¹⁸F]fluoride, [¹⁸F]deoxyfluorothymidine, [¹⁸F]fluoromisonidazole, [¹¹C]choline, or [¹¹C]methionine based scan.

18. The method of claim 16, further comprising:
processing image data of a pre treatment scan of the tissue of interest to determine whether the radiolabeled analog of compound 0118 is present in the tissue of interest represented in the image data; and
generating a first signal indicating that compound 0118 is a candidate treatment for the patient in response to the determining that the radiolabeled analog of compound 0118 is present in a predetermined amount in the tissue of interest represented in the image data.

19. The method of any of claim 18, further comprising:
generating a second signal indicating that compound 0118 is not a candidate treatment for the patient in response to the determining that the radiolabeled analog of compound 0118 is present in the predetermined amount in the tissue of interest represented in the image data.

20. A computing system, comprising:
a radiotracer identifier that processes at least one of PET or SPECT image data and identifies a presence or absence of a predetermined amount of radiolabeled analog of compound 0118 in tissue of interest of a patient represented in the image data; and
a recommender that generates and visually presents a first recommendation indicating that compound 0118 is a candidate treatment for the patient in response to the radiotracer identifier identifying the presence of the predetermined amount of radiolabeled analog of compound 0118 in tissue of interest of a patient represented in the image data.

21. The computing system of claim 20, wherein the recommender generates and visually presents a second recommendation indicating that compound 0118 is not a candidate treatment for the patient in response to the radiotracer identifier identifying the absence of the predetermined amount of radiolabeled analog of compound 0118 in tissue of interest of a patient represented in the image data.

22. The computing system of claim 20, wherein the radiotracer identifier identifies the presence of the predetermined amount of the radiolabeled analog of compound 0118 prior to a treatment of the patient with compound 0118.

23. The computing system of claim 20, wherein the radiotracer identifier identifies the presence or absence of the predetermined mount of the radiolabeled analog of compound 0118 during treatment of the patient with compound 0118.

24. The computing system of claim 23, wherein the recommender recommends continuing treatment with compound 0118 in response to the radiotracer identifier identifying the presence the predetermined amount of the radiolabeled analog of compound 0118 during treatment of the patient with compound 0118.

25. The computing system of claim 23, wherein the recommender recommends discontinuing treatment with compound 0118 in response to the radiotracer identifier identifying the absence of the predetermined amount of the radiolabeled analog of compound 0118 during treatment of the patient with compound 0118.

26. The computing system of claim 20, wherein the radiotracer identifier identifies at least one of an amount, a concentration or a distribution of the radiolabeled analog of compound 0118 in the tissue of interest of a patient represented in the image data and visually presents the identified at least one of the amount, the concentration or the distribution.

27. The computing system of claim 20, wherein the presence of the radiolabeled analog of compound 0118 in the tissue of interest indicates presence of a cancer having a galectin-1 molecular target, which is a cancer treatable by compound 0118.

28. A radiotracer, comprising:
an analog of compound 0118; and
a radiolabel.

29. The radiotracer of claim 28, wherein the radiotracer is therapeutic.

30. A radiolabeled analog of compound 0118, comprising:
a central calix[4]arene core; and
a hydrophobic substituent at the upper rim of the calix[4]arene,
wherein the substituent is one of a radioiodinated or a radiobrominated derivative accessible via radiohalodestannylation from a corresponding tributylstannyl precursor.

31. A radiolabeled analog of compound 0118, comprising:
a central calix[4]arene core; and
a hydrophilic substituent at a lower rim,
wherein the substituent is a radiolabel.

32. The radiolabeled analog of compound 0118 of claim 31, wherein the radiolabel includes an [¹⁸F]fluoroalkyltriazole moiety.

33. A radiolabeled analog of compound 0118 according to formula I: 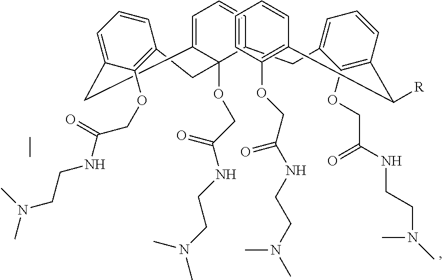
comprising:
a central calix[4]arene core; and
a substituent R at an equatorial position on a methylene bridge of the calix[4]arene core.

34. The radiolabeled analog of compound 0118 of claim 33, wherein the substituent R is from a group consisting of an [¹⁸F]fluoroalkyl chain or an [¹⁸F]fluoroalkyltriazole moiety.

35. A non-radioactive analogue of compound 0118 according to formula 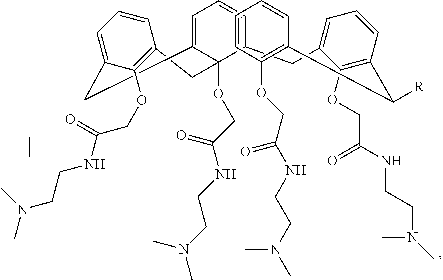
comprising:
a central calix[4]arene core; and
substituent R at an equatorial position on a methylene bridge of the calix[4]arene core.

36. The radiolabeled analog of compound 0118 of claim 35, wherein the substituent R is linear or branched alkyl, alkynyl, alkenyl cycloalkyl, heterocycloalkyl, aralkyl, heteroaralkyl, alkoxy, thioalkoxy, cycloaklalkoxy, or heterocycloalkylalkoxy.

37. The radiolabeled analog of compound 0118 of claim 36, wherein a group R includes halogen, hydroxyl, sulfhydryl, amide, ester, (poly)ether, phosphonate, sulfonate and/or heteroaryl.

38. A macromolecular structure containing two or more tethered moieties of a non-radioactive analogue of compound 0118 according to formula I: 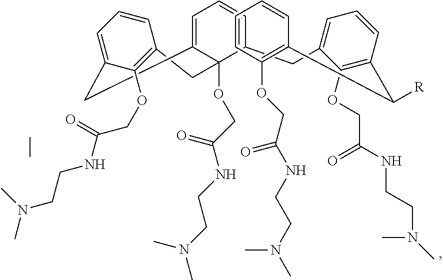
wherein the individual calix[4]arene subunits are attached to each other via a linker extending from the equatorial substituent R on the methylene bridge of the calix[4]arene core.

39. The macromolecular structure of claim 38, wherein the linker consists of branched and/or straight polyethylene(PEG)-chains of variable chain length, which in turn may be attached to a dendrimeric core structure and/or a suitable No-compatible polymer.

40. A macromolecular structure according to claim 38, wherein the linkers and/or polymeric support are made from a bio-degradable material allowing controlled release of therapeutically active modified compound 0118 entities, which includes both monomeric compound 0118 analogues according to formula I and therapeutically active di- and multimeric structures according to claims 35, 38, and 39.

41. Both non-radioactive and radiolabelled analogues of compound 0118 according to claim 33, wherein the application is therapeutic.

42. The analogues according to claim 41, wherein the analogues include nonradioactive analogues of the analogues of compound 0118.

43. A therapeutic method to inhibit angiogenesis in a patient, comprising administration of therapeutically effective amounts and formulations of analogues of compound 0118 according to claim 41.

44. The therapeutic method according to claim 43, wherein the therapeutic application is inhibition of tumorigenesis in a patient.

45. The therapeutic method according to claim 44, wherein the therapeutic application in patients is inhibition of diabetic retinopathy, inhibition of neovascular glaucoma, inhibition of rheumatoid arthritis, inhibition of restenosis, and inhibition of diabetic retinopathy.
